SEQUENCE LISTING (37 sequences)

SEQ ID NO: 1 - DNA, 1113 bp, synthetic construct
  source 1..1113: mol_type = other DNA
atggaagccaccaagcaagtggttaactttgggcccgggcctgccaagctgccacactcggtattgttggagatccagaagcagctactagactacagaggactcggcatcagtgtgctcgaaatgagtcacaggtcatcagattttgccaagattattggcaatacagagaatcttgtgagggaattgctagctgttcccaacaactacaaggtgatctttgtacaaggaggtgggtctggccagttcagtgctgtccccttaaatctgattggcctgaaagctggaaggagtgctgactacgtggtgaccggagcttggtcagctaaggctgcggaagaagccaagaagtttggaacggtgaacattgtccaccctaaacttggaagttacacaaaaattccagacccaagcacctggaacctcaacccggacgcctcctatgtatacttctgtgcaaacgagactgtgcacggggtggagtttgacttcgtacctgacgtcaagggagcggtgctggtctgtgacatgtcctcaaacttcttatccaggccggtggatgtttccaagtttggtgtgattttcgctggtgctcagaagaatgttggctctgccggggtgacggtggtgattgtccgggatgacctgctggggttctcgctcagagagtgcccatcagtccttgactacaaagtgcaggctgggaacaactctttgtataacacacctccgtgcttcagcatctacgtcatgggcatggtcctggaatggatcaagaacaacggcggggccgcagccatggagaagctcagctccatcaaatcccaaatgatttatgagatcattgataattctcaaggattttatgtatgcccagtggagcgccagaatagaagcaggatgaacatcccatttcgcattggcaacgccaaaggagacgaagctttggaaaagcggtttcttgacaaggcggtagaactcaacatgatctccttgaaggggcacaggtcagtgggaggcattcgtgcctctctgtataacgctgtcacaaccgaagacgttgagaagctggcggccttcatgaagaatttcttggagatgcatcagctgtga

SEQ ID NO: 2 - RNA, 1113 nt, synthetic construct
  source 1..1113: mol_type = other RNA
atggaagccaccaagcaagtggttaactttgggcccgggcctgccaagctgccacactcggtattgttggagatccagaagcagctactagactacagaggactcggcatcagtgtgctcgaaatgagtcacaggtcatcagattttgccaagattattggcaatacagagaatcttgtgagggaattgctagctgttcccaacaactacaaggtgatctttgtacaaggaggtgggtctggccagttcagtgctgtccccttaaatctgattggcctgaaagctggaaggagtgctgactacgtggtgaccggagcttggtcagctaaggctgcggaagaagccaagaagtttggaacggtgaacattgtccaccctaaacttggaagttacacaaaaattccagacccaagcacctggaacctcaacccggacgcctcctatgtatacttctgtgcaaacgagactgtgcacggggtggagtttgacttcgtacctgacgtcaagggagcggtgctggtctgtgacatgtcctcaaacttcttatccaggccggtggatgtttccaagtttggtgtgattttcgctggtgctcagaagaatgttggctctgccggggtgacggtggtgattgtccgggatgacctgctggggttctcgctcagagagtgcccatcagtccttgactacaaagtgcaggctgggaacaactctttgtataacacacctccgtgcttcagcatctacgtcatgggcatggtcctggaatggatcaagaacaacggcggggccgcagccatggagaagctcagctccatcaaatcccaaatgatttatgagatcattgataattctcaaggattttatgtatgcccagtggagcgccagaatagaagcaggatgaacatcccatttcgcattggcaacgccaaaggagacgaagctttggaaaagcggtttcttgacaaggcggtagaactcaacatgatctccttgaaggggcacaggtcagtgggaggcattcgtgcctctctgtataacgctgtcacaaccgaagacgttgagaagctggcggccttcatgaagaatttcttggagatgcatcagctgtga

SEQ ID NO: 3 - protein, 370 aa, synthetic construct
MEATKQVVNFGPGPAKLPHSVLLEIQKQLLDYRGLGISVLEMSHRSSDFAKIIGNTENLVRELLAVPNNYKVIFVQGGGSGQFSAVPLNLIGLKAGRSADYVVTGAWSAKAAEEAKKFGTVNIVHPKLGSYTKIPDPSTWNLNPDASYVYFCANETVHGVEFDFVPDVKGAVLVCDMSSNFLSRPVDVSKFGVIFAGAQKNVGSAGVTVVIVRDDLLGFSLRECPSVLDYKVQAGNNSLYNTPPCFSIYVMGMVLEWIKNNGGAAAMEKLSSIKSQMIYEIIDNSQGFYVCPVERQNRSRMNIPFRIGNAKGDEALEKRFLDKAVELNMISLKGHRSVGGIRASLYNAVTTEDVEKLAAFMKNFLEMHQL

SEQ ID NO: 4 - DNA, 1653 bp, synthetic construct
  source 1..1653: mol_type = other DNA
atggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaa

SEQ ID NO: 5 - DNA, 678 bp, synthetic construct
  source 1..678: mol_type = other DNA
atggtctcccactcagagctgaggaagctcttctgttcagcggatgcagtgtgctttgatgttgatagcaccgtcatcagagaagaaggaatcgatgagctggccaaattctgtggtgtggaggccgcagtgtctgaaatgacacggagagccatgggaggagcattgccttttaaagacgcgctcactcagcgcctggcactgatccagccctccagggatcaagtccagaggctcctagctgagcacccgccacatctgactcctggcataagggagctggtaagccgcctccaggagcgtaatgtccaggtgttcctcatctctggtggctttcggagcattgtggagcacgttgctgcaaagctcaatatcccaacaaccaatgtgtttgccaataggctgaagttctactttaatggtgagtacgcaggttttgatgagatgcagccgacagccgagtcgggtgggaaaggaaaggttattcggtttttaaaggaaaaatttcactttaagaaaataatcatgattggagatggagctacggacatggaagcctgtcctcctgctgatgctttcattggctttggaggcaatgtgatcaggcagcaggttaaggacaacgccaagtggtacatcactgacttcgtggagctgctgggagaactggaggagtga

SEQ ID NO: 6 - DNA, 1602 bp, synthetic construct
  source 1..1602: mol_type = other DNA
atggccttcgcaaatctgcgcaaagtactcatcagtgacagcctggacccctgctgccggaagatcctgcaagatggagggctgcaggtggtggagaagcagaacttgagcaaggaggagctgatagctgaactccaggactgtgaaggcctcattgtccggtctgctactaaggtcactgctgatgtcatcaatgcagcagagaagctccaggtggtgggcagggctggcacaggtgtggacaatgtggatctggaggctgccacgaggaagggcatcctagtcatgaacacccccaacgggaacagcctcagtgctgcggagctcacctgtgggatgatcatgtgcctggccaggcagattccccaggcaacagcttcgatgaaagatggcaaatgggaccggaagaagttcatggggacagaactgaacgggaagacactgggaattcttggcctcggcagaattggaagagaggtggccacccgaatgcaatcctttggaatgaagactgtaggctatgaccccatcatctctcctgaagtcgccgcctcctttggtgttcagcagctgccgctggaggagatctggcctctctgtgactttataactgtccataccccactcctgccctctaccacaggcttgctgaatgacagcacctttgctcagtgcaagaaaggtgtgcgagtagtgaactgtgctcgaggaggcattgtggatgaaggtgccctgctgcgtgccctgcagtctggtcagtgtgctggggctgcactggatgtgtttacagaagagccaccacgggaccgggccttagtggaccacgagaatgtcatcagctgtccccacctgggtgccagcaccaaggaagcccagagccgctgtggggaggaaatcgcagtccagtttgtggacatggtgaaggggaaatctctaacaggcgttgtgaacgcccaggccctcaccagtgccttctctccacacaccaagccttggattggtctggcagaagcaatgggcacgctgatgcacgcctgggctggctcccctaaagggaccatccaggtggttacacaaggaacatctctgaagaatgctgggacctgcctgagccctgcagtcattgtcggccttctgagagaagcatctaagcaggcagacgtgaacttggtgaacgctaagctactggtgaaagaggctggcctcaatgtcaccacctcccacaaccctggggttccaggggagcagggtagcggggaatgcctcctgactgtggccctggcaggtgctccctaccaagccgtgggcttggtccagggcaccacaccaatgctgcagatgctcaacggagctgtcttcagaccagaggtgccactacgcaggggccaacccctgctcgtattccgggctcagccctccgaccctgggatgctgcccactatgattggcctcctggcagaggcgggtgtacagctgctgtcctaccaaacctccatggtgtctgacggagagccctggcacgtcatgggcctgtcctccctgctgcccagcctggaaacatggaagcagcatgtattggaggctttccagttctgcttctga

SEQ ID NO: 7 - DNA, 23 bp, synthetic construct
  source 1..23: mol_type = other DNA
aggcacataggaggtaaaagttc

SEQ ID NO: 8 - DNA, 20 bp, synthetic construct
  source 1..20: mol_type = other DNA
ggaggtcacaggaggacttc

SEQ ID NO: 9 - DNA, 20 bp, synthetic construct
  source 1..20: mol_type = other DNA
ttggttctgtcggatgtctg

SEQ ID NO: 10 - DNA, 20 bp, synthetic construct
  source 1..20: mol_type = other DNA
ttggttctgtcggatgtctg

SEQ ID NO: 11 - DNA, 21 bp, synthetic construct
  source 1..21: mol_type = other DNA
catcctcccgagtgagtaatg

SEQ ID NO: 12 - DNA, 20 bp, synthetic construct
  source 1..20: mol_type = other DNA
gggtgaagaaagggctaaag

SEQ ID NO: 13 - DNA, 20 bp, synthetic construct
  source 1..20: mol_type = other DNA
catttgttccagagccagtc

SEQ ID NO: 14 - DNA, 20 bp, synthetic construct
  source 1..20: mol_type = other DNA
ttctttgggtgccttgtctc

SEQ ID NO: 15 - DNA, 20 bp, synthetic construct
  source 1..20: mol_type = other DNA
ccaggacagccactctcaag

SEQ ID NO: 16 - DNA, 20 bp, synthetic construct
  source 1..20: mol_type = other DNA
gcacaagagccaagattcag

SEQ ID NO: 17 - RNA, 21 nt, synthetic construct
  source 1..21: mol_type = other RNA
tagtcgtgggatcgcaccctg

SEQ ID NO: 18 - RNA, 19 nt, synthetic construct
  source 1..19: mol_type = other RNA
atgtccatgacgtagatgc

SEQ ID NO: 19 - RNA, 19 nt, synthetic construct
  source 1..19: mol_type = other RNA
aaagggatctgaactattg

SEQ ID NO: 20 - DNA, 20 bp, synthetic construct
  source 1..20: mol_type = other DNA
caagcacctggaacctcaac

SEQ ID NO: 21 - DNA, 20 bp, synthetic construct
  source 1..20: mol_type = other DNA
caccagcgaaaatcacacca

SEQ ID NO: 22 - DNA, 20 bp, synthetic construct
  source 1..20: mol_type = other DNA
aggcagctctggagtgagag

SEQ ID NO: 23 - DNA, 20 bp, synthetic construct
  source 1..20: mol_type = other DNA
cctggctcgcagattgtaag

SEQ ID NO: 24 - DNA, 21 bp, synthetic construct
  source 1..21: mol_type = other DNA
ttcgtgttcgagagcgatttg

SEQ ID NO: 25 - DNA, 22 bp, synthetic construct
  source 1..22: mol_type = other DNA
accttggaactagatttgccag

SEQ ID NO: 26 - DNA, 21 bp, synthetic construct
  source 1..21: mol_type = other DNA
tttcggccttgccagagcgtt

SEQ ID NO: 27 - DNA, 20 bp, synthetic construct
  source 1..20: mol_type = other DNA
gtggagtagcgagccgagcc

SEQ ID NO: 28 - DNA, 20 bp, synthetic construct
  source 1..20: mol_type = other DNA
gtcacccctcacgacttcat

SEQ ID NO: 29 - DNA, 20 bp, synthetic construct
  source 1..20: mol_type = other DNA
ttccagttgcaatcatcgac

SEQ ID NO: 30 - DNA, 20 bp, synthetic construct
  source 1..20: mol_type = other DNA
aaggtgcctgtgtgtgaacc

SEQ ID NO: 31 - DNA, 19 bp, synthetic construct
  source 1..19: mol_type = other DNA
gtcagccccatcatctgcg

SEQ ID NO: 32 - DNA, 21 bp, synthetic construct
  source 1..21: mol_type = other DNA
agtccctgccctttgtacaca

SEQ ID NO: 33 - DNA, 19 bp, synthetic construct
  source 1..19: mol_type = other DNA
cgatccgagggcctcacta

SEQ ID NO: 34 - DNA, 20 bp, synthetic construct
  source 1..20: mol_type = other DNA
aggagagccaggatgtcagc

SEQ ID NO: 35 - DNA, 21 bp, synthetic construct
  source 1..21: mol_type = other DNA
cagagtttgcctgagacccaa

SEQ ID NO: 36 - DNA, 20 bp, synthetic construct
  source 1..20: mol_type = other DNA
gacaagaggcccagtacttc

SEQ ID NO: 37 - DNA, 23 bp, synthetic construct
  source 1..23: mol_type = other DNA
gcttggagtgatagaaatctgtc

(19) United States
(12) Patent Application Publication
(10) Pub. No.: US 2025/0215403 A1
(43) Pub. Date: Jul. 3, 2025

(54) Compositions and Methods for Cardiac Tissue Regeneration

(71) Applicant: Temple University - Of The Commonwealth System Of Higher Education, Philadelphia, PA (US)

(72) Inventors: Raj KISHORE, Devon, PA (US); Ajit Magadum, Elkins Park, PA (US)

(21) Appl. No.: 18/836,775

(22) PCT Filed: Feb. 8, 2023
(86) PCT No.: PCT/US2023/062182

§ 371 Date: Aug. 8, 2024

Related U.S. Application Data
(60) Provisional application No. 63/307,960, filed on Feb. 8, 2022.

(51) Int. Cl. C12N 9/10; A61K 38/00; A61P 9/10

(52) U.S. Cl. CPC C12N 9/1096; A61P 9/10; C12Y 206/01052; A61K 38/00

(57) ABSTRACT

The present invention generally relates to novel compositions and methods for regenerating damaged cardiac tissue. In some embodiments, the invention comprises activators of phosphor-serine aminotransferase (PSAT1) and methods of use thereof for the treatment of damaged cardiac tissue following myocardial infarction.

### CROSS-REFERENCE TO RELATED APPLICATIONS

The present application claims priority to U.S. Provisional Application No. 63/307,960, filed on Feb. 8, 2022, which is incorporated by reference herein in its entirety.

### STATEMENT REGARDING FEDERALLY SPONSORED RESEARCH OR DEVELOPMENT

This invention was made with government support under Grant No. HL 134608 awarded by the National Institutes of Health. The government has certain rights in the invention.

### INCORPORATION BY REFERENCE OF A SEQUENCE LISTING IN XML FORMAT

The present application hereby incorporates by reference the entire contents of the XML file named “206017-0213-00WO_SequenceListing.xml” in XML format, which was created on Feb. 5, 2023, and is 39,929 bytes in size.

### BACKGROUND OF THE INVENTION

Permanent loss of cardiomyocytes (CM) after myocardial infarction (MI) and limited cardiac regenerative capacity leads to Heart Failure. iPSC-derived extracellular vesicles (EV) have been shown to improve cardiac function and protection. However, mechanism by which iPSC-EVs mediate cardiac function improvement remains unclear.

Mammalian CM proliferation rapidly ceases after birth, and the heart further grows by an increase in CM size compared to CM numbers. Adult zebrafish, newt, and even 1-day old neonatal mice can regenerate their heart through inducing existing CM proliferation post-injury. Previously, many attempts have been made to transiently reconstitute embryonic signaling in adult hearts, including overexpression of cell cycle activating genes or finding novel genes or molecular pathways with limited success. The inhibition of hippo pathway or YAP1 activation plays a major role in CM proliferation and cardiac regeneration. YAP1 is regulated at multiple levels in heart including transcriptional, post-translational and epigenetic mechanisms. However, not much is known about the role of YAP1 in cardiac metabolism-mediated cardiac regeneration. Thus, there is a need in the art for improved compositions and methods for the regeneration of cardiomyocytes (CMs) in the treatment of heart failure after myocardial infarction (MI). This invention satisfies this unmet need.

### SUMMARY OF THE INVENTION

In one embodiment, the present invention generally relates to a composition for regenerating cardiac tissue, comprising an activator of phospho-serine aminotransferase (PSAT1).

In one embodiment, said activator of PSAT1 comprises a nucleic acid molecule encoding PSAT1. In one embodiment, said nucleic acid molecule encoding PSAT1 comprise a modified RNA molecule. In one embodiment, said modified RNA molecule comprises N1-methylpseudouridine in place of naturally occurring uridine. In one embodiment, said modified RNA molecule comprises a codon optimized sequence for expression in mammalian cells. In one embodiment, said modified RNA molecule encoding PSAT1 comprises one or more selected from the group consisting of: a) an RNA sequence at least 90% identical to SEQ ID NO: 2; b) an RNA sequence at least 90% of the length of SEQ ID NO: 2; c) an RNA sequence at least 90% identical to a nucleotide sequence at least 90% of the length of SEQ ID NO: 2; and d) the RNA sequence of SEQ ID NO: 2.

In one embodiment, said nucleic acid molecule encoding PSAT1 comprises one or more selected from the group consisting of: a) a nucleotide sequence at least 90% identical to SEQ ID NO: 1; b) a nucleotide sequence at least 90% of the length of SEQ ID NO: 1; c) a nucleotide sequence at least 90% identical to a nucleotide sequence at least 90% of the length of SEQ ID NO: 1; and d) the nucleotide sequence of SEQ ID NO: 1. In one embodiment, said nucleic acid molecule comprises a plasmid vector. In one embodiment, said plasmid vector comprises a plasmid vector optimized for expression in mammalian cells.

In one embodiment, said activator of PSAT1 comprises a PSAT1 polypeptide. In one embodiment, said PSAT1 polypeptide comprises one or more selected from the group consisting of: a) an amino acid sequence at least 90% identical to SEQ ID NO: 3; b) an amino acid sequence at least 90% of the length of SEQ ID NO: 3; c) an amino acid sequence at least 90% identical to an amino acid sequence at least 90% of the length of SEQ ID NO: 3; and d) the amino acid sequence of SEQ ID NO: 3.

In one embodiment, the composition of the invention further comprises a pharmaceutically acceptable carrier.

In one embodiment, the present invention generally relates to a method of administering a composition for regenerating cardiac tissue to subject in need thereof, comprising administering to the subject a composition of the invention described herein.

In one embodiment, the present invention generally relates to a method of regenerating cardiac tissue in a subject in need thereof, comprising administering to the subject an activator of PSAT1.

In one embodiment, the present invention generally relates to a method of treating a disease or disorder associated with cardiac tissue damage in a subject in need thereof, comprising administering to the subject an activator of PSAT1. In one embodiment, the method further comprises administering one or more additional therapies for the disease or disorder. In one embodiment, disease or disorder comprises one or more selected from the group consisting of: fibrotic diseases, myocardial infarction, ischemic heart disease, heart failure, and dilated cardiomyopathy (DCM).

In some embodiments of the methods described herein, said activator of PSAT1 is administered via intra-myocardial injection. In some embodiments, said intra-myocardial injection is localized to the peri-infarct area of the infarcted heart.

In some embodiments of the methods described herein, the subject is a mammal. In some embodiments, the subject is a human.

In some embodiments of the methods described herein, the gene expression of PSAT1 increases after administration of the activator of PSAT1. In some embodiments, gene expression of PSAT1 increases by at least 10-fold one day after administration of the activator of PSAT1.

In one embodiment, the present invention generally relates to a method for inducing/reactivating proliferation of neonatal or adult cardiomyocytes in vitro or in vivo following myocardial infarction (MI). In one embodiment, the method comprises myocardially administering one or more modRNA selected from the group consisting of phosphoglycerate dehydrogenase (PHGDH), PSAT1, and phosphoserine phosphatase (PSPH), wherein the modRNA induces the cardiomyocyte cell cycle and inhibits cardiomyocyte apoptosis at least seven days post-MI.

In one embodiment, the present invention generally relates to a method for inducing or activating the serine metabolic pathway of neonatal or adult cardiomyocytes in vitro or in vivo following myocardial infarction, comprising administering an activator of PSAT1. In one embodiment, activation of the SSP inhibits cardiomyocyte apoptosis and oxidative stress post-MI.

In one embodiment, the present invention generally relates to a method for inhibiting oxidative stress and reactive oxygen species in neonatal or adult cardiomyocytes in vitro or in vivo following myocardial infarction, comprising administering an activator of PSAT1.

In one embodiment, the present invention generally relates to a method for inhibiting cardiomyocyte apoptosis in neonatal or adult cardiomyocytes in vitro or in vivo following myocardial infarction, comprising administering an activator of PSAT1.

In one embodiment, the present invention generally relates to a method for stabilizing β-catenin and its translocation to the nucleus in neonatal cardiomyocytes in vitro, comprising administering an activator of PSAT1.

In one embodiment, the present invention generally relates to a method of promoting activation of PSAT1 by YAP1 through transactivation in neonatal cardiomyocytes in vitro, comprising administering one or more composition that promotes YAP1 binding to the promoter site of PSAT1, thereby inducing expression of PSAT1.

In one embodiment, the present invention generally relates to a method for inducing nucleotide synthesis in neonatal or adult cardiomyocytes in vitro or in vivo following myocardial infarction, comprising administering an activator of PSAT1.

In one embodiment, the present invention generally relates to a method of treating ischemic heart injury in a subject in need thereof, the method compromising administering a modified mRNA (modRNA) encoding phosphoserine aminotransferase 1 or phosphohydroxythreonine aminotransferase 1 (PSAT1) to a heart tissue of the subject. In one embodiment, administering the modRNA encoding PSAT1 to heart tissue of a subject in need thereof improves heart function by at least 50% compared to an untreated subject with identical disease condition and predicted outcome. In one embodiment, administering the modRNA encoding PSAT1 to heart tissue of a subject in need thereof increases life expectancy by at least 20% compared to an untreated subject with identical disease condition and predicted outcome. In one embodiment, administering the modRNA encoding PSAT1 to heart tissue of a subject in need thereof reduces cardiac fibrosis by at least 50% compared to an untreated subject with identical disease condition and predicted outcome.

In one embodiment, the present invention generally relates to a gene delivery system for treatment of ischemic heart injury, comprising modRNA encoding PSAT1 for local administration to heart tissue. In one embodiment, the modRNA encoding PSAT1 increases PSAT1 expression in said heart tissue. In one embodiment, the gene delivery system further compromises a delivery agent. In one embodiment, the delivery agent specifically targets hearts tissue. In one embodiment, the gene delivery system is formulated for intracardiac injections.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1, comprising FIG. 1A through FIG. 1F, depict representative improvements in post-myocardial (post-MI) cardiac functions and induced cardiomyocyte (CM) cell cycle. FIG. 1A depicts a schematic representation of an experimental timeline to evaluate cardiac function and CM cell cycle in a MI mouse model 28- or 7-days post-MI. FIG. 1B depicts representative results of echo evaluation of % EF in PBS, hiPSC-EVs or IMR90-EVs treated mice. FIG. 1C depicts representative results of echo evaluation of % FS 28 post-MI. FIG. 1D depicts representative images of ki67⁺ (Green) or pH3⁺ (Green), CM-specific marker Actinin⁺ (Red) and DAPI (blue) seven days post-MI and EV injection. Scale bar=100 μm. FIG. 1E depicts representative quantification of Ki67 cell cycle markers of post-MI CMs when treated with PBS, IMR90 EVs, or HiPSC EVs. FIG. 1F depicts representative quantification of pH3 cell cycle markers of post-MI CMs when treated with PBS, IMR90 EVs, or HiPSC EVs. One-way ANOVA for FIGS. 1B, 1C, 1E, and 1F; ***, P<0.001; **, P<0.01*; P<0.05.

FIG. 2, comprising FIGS. 2A through 2H, depicts proteomic results demonstrating that PSAT1 is a hiPSC-EV protein that is down-regulated during mouse heart development. FIG. 2A depicts a schematic representation of proteomic analysis of EVs from hiPSC or IMR90. FIG. 2B depicts a representative heat map of proteomic analysis showing differential expression of proteins between hiPSC or IMR90 derived EVs by mass spectrometry analysis. FIG. 2C depict a representative Venn diagram of differentially expressing proteins between hiPSC or IMR90-EVs. FIG. 2D depicts representative proteomic analysis showing PSAT1 protein exclusively expressed in hiPSC EVs. FIG. 2E depicts a representative image of Western-blot analysis of PSAT1 protein expression between hiPSC or IMR90 derived EVs. FIG. 2F depicts representative quantitative analysis of PSAT1 protein expression depicted in FIG. 2E (n=2). FIG. 2G depicts a representative QPCR analysis of PSAT1 mRNA expression during mouse heart development (n=3). FIG. 2H depicts representative images of PSAT1 expression (red), CM-specific marker Actinin⁺ (green), and DAPI (blue) in mouse myocardia during heart development (n=3). Unpaired two-tailed t-test for FIGS. 1D and 1F or one-way ANOVA for FIG. 1G. ***, P<0.001; **, P<0.01*; P<0.05.

FIG. 3, comprising FIG. 3A through FIG. 3D, depicts representative results of proteomic analysis of hiPSC- or IMR90-derived EVs. FIG. 3A depicts a representative quantitative analysis of HiPSC- or IMR90-EV specific proteins. FIG. 3B depicts representative depiction of proteins highly expressed only in hiPSC-EVs relative to IMR90 EVs. FIG. 3C depicts representative expression of the top 19 nonstructural proteins expressed in hiPSC-EVs over IMR90-EVs. FIG. 3D depicts quantification of PSAT1 mRNA expression in HiPSC-vs IMR90-EVs (n=3); unpaired two-tailed t-test; ***, p<0.001.

FIG. 4, comprising FIG. 4A through FIG. 4C, depicts representative PSAT1 expression post-MI. FIG. 4A depicts a schematic representation of an experimental setup for PSAT1 mRNA or protein expression in sham vs MI mouse model. FIG. 4B depicts representative quantification of PSAT1 mRNA expression 2 days post-sham or post-MI (n=3); unpaired two-tailed t-test; *, p<0.05. FIG. 4B depicts representative images of PSAT1 expression (white arrow showing CMs, yellow arrow showing PSAT1 expression in non-CMs) with red (PSAT1+) or green (α sarcomeric actinin⁺) CM-specific markers and DAPI-nucleus markers; scale=20 μm.

FIG. 5, comprising FIG. 5A through FIG. 5H, depicts representative experimental results demonstrating that PSAT1 modRNA induces cardiomyocyte cell cycle in vitro and in vivo. FIG. 5A depicts a schematic representation of an experimental setup for PSAT1 modRNA expression in NRVM and its effect on CM cell cycle in vitro. FIG. 5B depicts representative images of PSAT1 expression 24 hours post-transfection of Luc or PSAT1 modRNA; red, PSAT1+; green alpha sarcomeric actinin⁺; blue, DAPI; scale bar=50 μm FIG. 5C depicts representative increase in relative PSAT1 protein expression in PSAT1 modRNA-treated cells analyzed by the immunostaining in FIG. 5B (n=4). FIG. 5D depicts representative images of PSAT1-induced CM mitosis analyzed by mitosis marker (pH3) expression (green), actinin⁺ (red), and DAPI (blue); scale bar=20 μm. FIG. 5E depicts representative quantification of pH3⁺ CMs in FIG. 5D (n=4). FIG. 5F depicts representative quantification of PSAT1-induced cardiomyocyte number 5 days post-transfection of Luc or PSAT1 modRNA expression (n=4). FIG. 5G depicts a schematic representation of an experimental setup for examining the effect of PSAT1 or Luc modRNA delivery on cell cycle marker expression 2 days post-MI. FIG. 5H depicts representative mRNA expression of cell-cycle-promoting genes or cell-cycle inhibitors after PSAT1 or Luc modRNA delivery post-MI (n=2-3). For FIGS. 5C, 5E, 5F, and 5H unpaired two-tailed t-test; ***, P<0.001; **, P<0.01.

FIG. 6, comprising FIG. 6A through FIG. 6M, depicts representative experimental results demonstrating that PSAT1 modRNA induces CM proliferation post-MI. FIG. 6A depicts a schematic representation of an experimental design for PSAT1 modRNA expression in myocardia post-MI and its examining the effect on CM cell cycle marker expression. FIG. 6B depicts representative images of PSAT1 modRNA expression (red) 24 hours post-MI after Luc or PSAT1 modRNA injection. FIG. 6C depicts representative images of BrdU⁺ (green), actinin⁺ (red), and DAPI (blue) in cardiomyocytes post-MI. FIG. 6D depicts representative images of pH3 (green), actinin⁺ (red), and DAPI (blue) in cardiomyocytes post-MI. FIG. 6E depicts representative images of Aurora B (green), troponin 1 (red), and DAPI (blue) in cardiomyocytes post-MI. FIG. 6F depicts representative quantification of the cell cycle marker BrdU in cells treated with Luc or PSAT1 modRNA. FIG. 6G depicts representative quantification of the cell cycle marker pH3 in cells treated with Luc or PSAT1 modRNA. FIG. 6H depicts representative quantification of the cell cycle marker Aurora B in cells treated with Luc or PSAT1 modRNA. FIG. 6I depicts a schematic representation of an experimental timeline to trace proliferating CMs using α-MHC-MADM mice. FIG. 6J depicts representative images of single-color-(green, eGFP⁺/DsRed⁻ or red, eGFP⁻/DsRed⁺) or double-color-(yellow, eGFP⁺/DsRed⁺) labeled CMs 28 days post-MI and injection of Luc or PSAT1 modRNA in α-MHC-MADM mice; scale bar=100 μm. FIG. 6K depicts representative quantification of single-color CMs (red or green) amongst total labeled CMs 28 days pos-MI. FIG. 6L depicts a representative distribution of single-color CMs in the heart 28 days post MI (infarct, border, or remote area). FIG. 6M depicts representative quantification of CM nucleation positive for single color (mono-, bi-, or multi-nuclear) 28 days post MI. For FIGS. 6F-6H and 6K-6M, n=5; unpaired two-tailed t-test; ***, P<0.001; **, P<0.01; *, P<0.05. For FIGS. 6C and 6D, scale bar=50 μm. For FIGS. 6E and 6F, scale bar=25 μm.

FIG. 7, comprising FIG. 7A and FIG. 7K, depicts representative experimental results demonstrating that PSAT1 modRNA delivery improves cardiac function post-MI. FIG. 7A depicts a schematic representation of an experimental timeline to evaluate cardiac function in MI mouse model 28 days post-MI. FIG. 7B depicts representative echo evaluation of percentage of ejection fraction (% EF). FIG. 7C depicts representative echo evaluation of percentage fractioning shorting (% FS) post-MI with Luc or PSAT1 modRNA. FIG. 7D depicts representative imaging of Masson trichrome staining to evaluate scar size 28 days post-MI with Luc or PSAT1 modRNA injection; scale bar=200 μm. FIG. 7E depicts representative quantification of scar size 28 days post-MI with Luc or PSAT1 modRNA injection. FIG. 7F depicts representative heart weight to body weight ratio 28 days post-MI with Luc or PSAT1 modRNA injection. FIG. 7G depicts representative images of WGA staining of heart after treatments with Luc or PSAT1 modRNA 28 days post-MI. FIG. 7H depicts representative CM size quantified by WGA in FIG. 7G. FIG. 7I depicts representative images of CD31⁺ staining of heart tissue after Luc or PSAT1 modRNA expression. FIG. 7J depicts representative quantification of capillaries in a 145 mm² tissue section. FIG. 7K depicts a representative long-term survival curve post-MI for mice injected with Luc or PSAT1 modRNAs, (n=10). For FIGS. 7B, 7C, 7E, 7F, 7H, and 7J, n=8 for Luc modRNA, n=9 for PSAT1 modRNA; unpaired two-tailed t-test; ****, P<0.0001; ***, P<0.001; **, P<0.01; *, P<0.05. For FIGS. 7G and 7I, scale bar=100 μm.

FIG. 8, comprising FIG. 8A through FIG. 8F, depicts representative results demonstrating myocardial delivery of PSAT1 modRNA improves cardiac function post-MI. FIG. 8A depicts a schematic representation of an experimental timeline used for evaluating the effect of PSAT1 or Luc modRNA delivery on cardiac function and outcome in a mouse MI model. FIG. 8B depicts representative echocardiography images of mouse left ventricle 28 days post-MI with Luc or PSAT1 modRNA delivery to the heart. FIG. 8C depicts representative echocardiography quantification of left ventricular internal diameter end-diastole (LVIDd). FIG. 8D depicts representative echocardiography quantification of left ventricular internal diameter end-systole (LVIDs). FIG. 8E depicts representative echocardiography quantification of end-diastolic left ventricular posterior wall thickness (LPVWd). FIG. 8F depicts representative echocardiography quantification of end-systolic left ventricular posterior wall thickness (LVPWs). For FIGS. 8C-8F, n=8-9; unpaired two-tailed t-test; ****, <0.0001; ***, P<0.001; *, P<0.05.

FIG. 9, comprising FIG. 9A through FIG. 9J, depicts representative regulation of CM proliferation and apoptosis by the serine synthesis pathway (SSP). FIG. 9A depicts a schematic representation of an experimental timeline used to evaluate ¹³C labeling of metabolites when fed ¹³C₆-glucose. FIG. 9B depicts representative results of intracellular labeled metabolites with glucose flux by mass spectrometry in P2-P3 NRVMs transfected with Luc or PSAT1 modRNA. Levels of ¹³C-labeled glycolysis metabolites were evaluated 10 min after glucose addition. TCA metabolites were evaluated 6 hours after ¹³C-glucose addition. Serine and nucleotide synthesis were evaluated 24 hours after ¹³C-glucose addition; n=2, unpaired two-tailed t-test. FIG. 9C depicts a schematic representation of an experimental plan for inhibition of SSP by PHGDH inhibitor (PH755, 10 μM) in presence or absence of PSAT1 modRNA in vitro. FIG. 9D depicts a schematic representation of serine metabolism showing that PH755 treatment impedes CM cell cycle by inhibiting SSP. FIG. 9E depicts representative quantitative analysis of serine levels in cell lysates (n=5). FIG. 9F depicts representative quantitative analysis of CM cell cycle by ki67⁺ CMs. FIG. 9G depicts representative quantitative analysis of CM cell cycle by pH3⁺ CMs. FIG. 9H depicts representative quantitative analysis of CM cell cycle by CM number. FIG. 9I depicts a schematic representation of an experimental timeline for TUNEL studies in NRVMs treated with or without PH755 in the presence or absence of PSAT1 modRNA and cells under hypoxic conditions for 24 hours. FIG. 9J depicts representative quantitative analysis of TUNEL⁺ CMs as outlined in FIG. 9I. For FIGS. 9E-9H and 9J, one-way ANOVA. For FIGS. 9B, 9D-9H, and 9J, ***, P<0.001; **, P<0.01; *, P<0.05.

FIG. 10, comprising FIG. 10A and FIG. 10B, depicts representative results demonstrating that delivery of PSAT1 modRNA into CMs redirects glucose carbon flow into serine and nucleotide biosynthetic pathways. FIG. 10A depicts representative quantification of intracellular labeled metabolites with glucose flux by mass spectrometry in P2-P3 NRVMs transfected with Luc or PSAT1 modRNA. Levels of ¹³C-labeled glycolysis metabolites were evaluated 10 min after glucose addition. TCA metabolites were evaluated 6 hours after ¹³C-glucose addition. Serine and nucleotide synthesis were evaluated 24 hours after ¹³C-glucose addition; n=2; unpaired two-tailed t-test; *, p<0.05. FIG. 10B depicts a schematic representation of PSAT1-induced modification of the metabolic pathway in neonatal cardiomyocytes.

FIG. 11, comprising FIG. 11A through FIG. 11E, depicts representative results demonstrating that PSAT1 modRNA increases serine and glycine levels in the hearts of mice post-MI. FIG. 11A depicts a schematic representation of an experimental timeline for evaluating the effect of Luc or PSAT1 modRNA delivery on serine and glycine levels post-MI. FIG. 11B depicts representative quantification of serine levels two days post-MI with Luc or PSAT1 modRNA. FIG. 11C depicts representative quantification of glutamic acid levels two days post-MI with Luc or PSAT1 modRNA. FIG. 11D depicts representative quantification of glycine levels two days post-MI with Luc or PSAT1 modRNA. FIG. 11E depicts representative quantification of L-glutamine levels two days post-MI with Luc or PSAT1 modRNA. For FIGS. 11B-11E, n=4; unpaired two-tailed t-test; **, P<0.01; *, P<0.05.

FIG. 12, comprising FIG. 12A through FIG. 12N, depicts representative results demonstrating that PSAT1 modRNA inhibits CM oxidative stress and apoptosis post-MI. FIG. 12A depicts a schematic representation of an experimental timeline used for evaluating the effect of PSAT1 or Luc modRNA delivery on CM apoptosis in a mouse MI model using the TUNEL method. FIG. 12B depicts representative images of TUNEL staining 2- or 7-days post-transfection of Luc or PSAT1 modRNA injection (red, TUNEL⁺; green, α-sarcomeric actinin⁺; blue, DAPI); scale bar=50 μm. FIG. 12C depicts representative quantitative analysis of TUNEL⁺ CMs from FIG. 12B 2 days post-MI. FIG. 12D depicts representative quantitative analysis of TUNEL⁺ CMs from FIG. 12B 7 days post-MI; n=5. FIG. 12E depicts a schematic representation of an experimental timeline used for quantification of ROS. FIG. 12F depicts representative HPLC quantification of ROS by superoxide probe dihydro-ethidium (DHE) in 2-hydroxyethidium (EOH) two days post-MI with Luc or PSAT1 modRNA. FIG. 12G depicts representative quantification of ROS by DHE in ethidium (E) two days post-MI with Luc or PSAT1 modRNA. FIG. 12H depicts representative quantification of the ratio of GSH to GSSG (reduced/oxidized glutathione) post-MI and modRNA delivery. FIG. 12I depicts a schematic representation of an experimental timeline used to evaluate the effect of Luc or PSAT1 modRNA delivery on DNA damage response in CMs in a mouse MI model. FIG. 12J depicts a schematic representation of DNA damage and base modification by ROS. FIG. 12K depicts representative images of 8-hyrdroxyguanosine (8-OHG) foci frequency in CMs 2 days after MI and transfection with either Luc or PSAT1 modRNA (co-stained for α-actinin and DAPI). FIG. 12L depicts a representative quantitative analysis of 8-OHG depicted in FIG. 12K. FIG. 12M depicts representative images of phosphorylation of ataxia telangiectasia mutated (pATM) foci frequency in CMs 2 days after MI and transfection with Luc or PSAT1 modRNA. FIG. 12N depicts representative quantitative analysis of pATM in FIG. 12M. For FIGS. 12C, 12L, and 12N, n=4. For FIGS. 12F-12H, n=4-5. For FIGS. 12C, 12D, 12F-12H, 12L, and 12N, unpaired two-tailed t-test; ***, P<0.001, **, P<0.01. For FIGS. 12K and 12M, scale bar=5 μm.

FIG. 13, comprising FIG. 13A through FIG. 13C, depicts representative experimental results demonstrating that PSAT1 modRNA inhibits oxidative stress post-MI. FIG. 13A depicts a schematic representation of an experimental timeline used for evaluating the effect of Luc or PSAT1 modRNA delivery on the levels of reducing agents NADH and NADPH. FIG. 13B depicts representative quantification of the ratio of NADH to NAD two days post-MI when treated with Luc or PSAT1 modRNA. FIG. 13C depicts representative quantification of the increase in NADPH two days post-MI when treated with Luc or PSAT1 modRNA. For FIGS. 13B and 13C, n=4; unpaired two-tailed t-test.

FIG. 14, comprising FIG. 14A through FIG. 14O, depicts representative experimental results demonstrating that the YAP1-PSAT1-β-catenin axis induces the CM cell cycle. FIG. 14A depicts a schematic diagram showing that YAP1 binds to the PSAT1 promoter sites in NRVMs. FIG. 14B depicts representative quantitative analysis of YAP binding to the promoter region of PSAT1 as shown in A. FIG. 14C depicts representative quantitative analysis showing PSAT1 mRNA levels after Luc or YAP1 modRNA expression in NRVMs. FIG. 14D depicts a schematic representation of an experimental timeline to study the effect of PSAT1 inhibition (by siRNA) on YAP1 induced CM cell cycle in NRVMs (Knock-down of PSAT1 inhibit YAP1 modRNA induced CM cell cycle). Cells were fixed 3 days post-transfection (siRNA and/or modRNA) and immunostained for pH3 antibody and actinin. FIG. 14E depicts representative images of PSAT1 inhibition 3 days post-transfection of scrambled or PSAT1 siRNA transfection (red, PSAT1+; green α-sarcomeric actinin⁺; blue, DAPI). FIG. 14F depicts representative quantitative analysis of FIGS. 14D and 14E for pH3⁺ CMs. FIG. 14G depicts representative quantitative analysis of CM number. FIG. 14H depicts representative images of Western-blot analysis of PSAT1 induced phosphorylation of Ser9 of GSK-3β in NRVMs compared to Luc modRNA. FIG. 14I depicts representative quantitative analysis of differential protein expression in FIG. 14G. FIG. 14J depicts representative images of β-catenin in CM nucleus and cytoplasm 48 hours after PSAT1 modRNA (co-stained α-actinin and DAPI). FIG. 14K depicts representative quantitative analysis of nuclear β-catenin in CMs. FIG. 14L depicts a schematic representation of an experimental timeline to study the effect of β-catenin inhibition (by siRNA) on PSAT1 induced CM cell cycle in NRVMs. Cells were fixed after 3 days post-transfection (siRNA and/or modRNA) and immunostained for ki67 or pH3 antibody. FIG. 14M depicts representative images of β-catenin inhibition 3 days post-transfection of scrambled or β-catenin siRNA transfection (red, β-catenin⁺; green, α-sarcomeric actinin⁺; blue DAPI). FIG. 14N depicts representative quantitative analysis of pH3⁺ in CMs of FIG. 14M; scale bar=50 μm. FIG. 14O depicts representative quantitative analysis of CM number. For FIGS. 14E and 14J, scale bar=25 μm. For FIGS. 14B and 14I, n=2. For FIGS. 14C, 14F, 14G, 14K, 14N, and 14O, n=2. For FIGS. 14B, 14C, 14I, and 14K, unpaired two-tailed t-test. For FIGS. 14F, 14G, 14N, and 14O, one-way ANOVA. ***, P<0.001; **, P<0.01; *, P<0.05.

FIG. 15, comprising FIG. 15A through FIG. 15E, depicts representative experimental results that YAP1 inhibition decreases PSAT1-induced CM cell cycle post-MI. FIG. 15A depicts a schematic representation of an experimental timeline to study the effect of YAP1 inhibition on PSAT1 induced CM cell cycle in the heart, AAV9-GFP or AAV9-YAP1-shRNA was retro-orbitally injected in mice 7 days before LAD ligation and PSAT1 modRNA delivery. Hearts were harvested 7 days post-MI for immunostaining. FIG. 15B depicts representative quantitative analysis showing YAP1 mRNA expression in mice hearts post shRNA inhibition; n=3; unpaired two-tailed t-test. FIG. 15C depicts representative images of CMs 7 days post-MI (green, pH3⁺; red, Troponin T⁺; blue, DAPI); scale bar=100 μm. FIG. 15D depicts representative quantitative analysis of pH3⁺ CMs in FIG. 15C; n=3-6; one-way ANOVA. FIG. 15E depicts a schematic representation of the PSAT1 induced molecular and metabolic signaling pathway in cardiac regeneration. ****, P<0.0001; ***, P<0.001; **, P<0.01; *, P<0.05.

FIG. 16, comprising FIG. 16A through FIG. 16D, depicts representative results demonstrating that SSP genes, alone or in combination, induce CM cell cycle and inhibit CM apoptosis. FIG. 16A depicts a schematic representation of an experimental timeline to study the effect of SSP gene (serine synthesis enzymes PHDGH, PSAT1, and PSPH, alone or in combination) modRNA expression on CM cell cycle and apoptosis post-MI. Tissues were harvested 7 days post-MI and immunostained for pH3 antibody, TUNEL levels, and serine levels were analyzed FIG. 16B depicts representative quantification of serine levels post SSP modRNA delivery analyzed using a calorimetric kit. FIG. 16C depicts representative quantitative analysis for pH3⁺ CMs post SSP modRNA delivery in mouse MI model. FIG. 16D depicts representative quantitative analysis for TUNEL⁺ CMs post SSP modRNA delivery (n=3). For FIGS. 16B-16D, n=3; unpaired two-tailed t-test; ****, P<0.0001; ***, P<0.001; **, P<0.01; *, P<0.05.

### DETAILED DESCRIPTION

The present invention generally relates to improved compositions for promoting cardiac regeneration following injury and for methods of treating cardiac injury following myocardial infarction.

The present invention is based, in part, upon the discovery that expression of phosphoserine aminotransferase (PSAT1), a protein that is exclusively expressed in human inducible pluripotent stem cell-derived extracellular vesicles (iPSC-EVs) and highly expressed during embryonic heart development, robustly induces CM proliferation via the YAP1-β-catenin pathway, inhibits apoptosis, oxidative stress and DNA damage, and reactivates cardiac regeneration post-MI. Thus, compositions and methods for introducing PSAT1 or inducing its expression represent novel therapies for cardiomyocyte regeneration post-MI.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “activate,” as used herein, means to induce or increase an activity or function, for example, about ten percent relative to a control value. Preferably, the activity is induced or increased by 50% compared to a control value, more preferably by 75%, and even more preferably by 95%. “Activate,” as used herein, also means to increase a molecule, a reaction, an interaction, a gene, an mRNA, and/or a protein's expression, stability, function or activity by a measurable amount or to increase entirely. Activators are compounds that, e.g., bind to, partially or totally induce stimulation, increase, promote, induce activation, activate, sensitize, or up regulate a protein, a gene, and an mRNA stability, expression, function and activity, e.g., agonists.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The terms “patient,” “subject,” “individual,” and the like are used interchangeably herein, and refer to any animal or cell whether in vitro or in vivo, amenable to the methods described herein.

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In one embodiment, the first region comprises a first portion and the second region comprises a second portion, whereby, when the first and second portions are arranged in an antiparallel fashion, at least about 50%, at least about 75%, at least about 90%, or at least about 95% of the nucleotide residues of the first portion are capable of base pairing with nucleotide residues in the second portion. In one embodiment, all nucleotide residues of the first portion are capable of base pairing with nucleotide residues in the second portion.

The term “DNA” as used herein is defined as deoxyribonucleic acid.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

The term “homology” refers to a degree of complementarity. There may be partial homology or complete homology (i.e., identity). Homology is often measured using sequence analysis software (e.g., Sequence Analysis Software Package of the Genetics Computer Group. University of Wisconsin Biotechnology Center. 1710 University Avenue. Madison, Wis. 53705). Such software matches similar sequences by assigning degrees of homology to various substitutions, deletions, insertions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

The term “inhibit,” as used herein, means to suppress or block an activity or function, for example, about ten percent relative to a control value. Preferably, the activity is suppressed or blocked by 50% compared to a control value, more preferably by 75%, and even more preferably by 95%. “Inhibit,” as used herein, also means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The term “nucleic acid molecule” refers to any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides. Conventional notation is used herein to describe polynucleotide sequences: the left-hand end of a single-stranded polynucleotide sequence is 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “operably linked” as used herein refer to the linkage of nucleic acid sequences in such a manner that a nucleic acid molecule capable of directing the transcription of a given gene and/or the synthesis of a desired protein molecule is produced. The term also refers to the linkage of sequences encoding amino acids in such a manner that a functional (e.g., enzymatically active, capable of binding to a binding partner, capable of inhibiting, etc.) protein or polypeptide is produced.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in an inducible manner.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “RNA” as used herein is defined as ribonucleic acid.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The terms “treat,” “treating,” and “treatment,” refer to therapeutic or preventative measures described herein. The methods of “treatment” employ administration to a subject, in need of such treatment, a composition of the present invention, for example, a subject afflicted a disease or disorder, or a subject who ultimately may acquire such a disease or disorder, in order to prevent, cure, delay, reduce the severity of, or ameliorate one or more symptoms of the disorder or recurring disorder, or in order to prolong the survival of a subject beyond that expected in the absence of such treatment.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

### Compositions

In one aspect, the present invention comprises novel compositions for use in regenerating cardiac tissue. In one embodiment, said cardiac tissue is damaged cardiac tissue. In one embodiment, the composition can be used to treat one or more disease or disorder associated with damaged cardiac tissue. In one embodiment, said disease or disorder comprises myocardial infarction. In one embodiment, said composition comprises an activator of a protein that is exclusively expressed in inducible human pluripotent stem cell-derived extracellular vesicles (hiPSC-EVs) but not in EVs from terminally differentiated cells. In one embodiment, said protein is highly expressed during embryonic heart development but expressed only at very low levels in adult heart. In one embodiment, said protein robustly induces cardiomyocyte (CM) proliferation. In one embodiment, said protein inhibits CM apoptosis, oxidative stress and DNA damage. In one embodiment, said protein comprises a phosphohydroxythreonine aminotransferase (PSAT1) polypeptide. In one embodiment, said composition comprises a nucleic acid encoding a PSAT1 polypeptide.

### Activators

In various embodiments, the composition for treating damaged cardiac tissue comprises an activator of PSAT1. In one embodiment, the activator of the invention increases the amount of PSAT1 polypeptide, the amount of PSAT1 mRNA, the level of PSAT1 activity, or any combination thereof.

It will be understood by one skilled in the art, based upon the disclosure provided herein, that an increase in the level of PSAT1 encompasses the increase in PSAT1 expression, including transcription, translation, or both. The skilled artisan will also appreciate, once armed with the teachings of the present invention, that an increase in the level of PSAT1 includes an increase in PSAT1 activity (e.g., an enhanced ability to stimulate proliferation, to promote serine biosynthetic pathways, to inhibit oxidative stress, etc.) Thus, increasing the level or activity of PSAT1 includes, but is not limited to, increasing the amount of PSAT1 polypeptide, increasing transcription, translation, or both, of a nucleic acid encoding PSAT1; and it also includes increasing any activity of a PSAT1 polypeptide as well.

Activation of PSAT1 can be assessed using a wide variety of methods, including those disclosed herein, as well as methods well-known in the art or to be developed in the future. That is, the skilled artisan would appreciate, based upon the disclosure provided herein, that increasing the level or activity of PSAT1 can be readily assessed using methods that assess the level of a nucleic acid encoding PSAT1 (e.g., mRNA) and/or the level of PSAT1 polypeptide in a biological sample obtained from a subject.

A PSAT1 activator can include, but should not be construed as being limited to, a chemical compound, a polypeptide, a peptidomimetic, an antibody, and a nucleic acid molecule. One of skill in the art would readily appreciate, based on the disclosure provided herein, that a PSAT1 activator encompasses a chemical compound that increases the level, activity, or the like of PSAT1. Additionally, a PSAT1 activator encompasses a chemically modified compound, and derivatives, as is well known to one of skill in the chemical arts.

One of skill in the art will further realize that diminishing the amount or activity of a molecule that itself inhibits the amount or activity of PSAT1 can serve to increase the amount or activity of PSAT1. Any inhibitor of a negative regulator of PSAT1 activity and/or expression is encompassed in the invention. As a non-limiting example, antisense DNA is described as a form of inhibiting a regulator of PSAT1 in order to increase the amount or activity of PSAT1. Antisense oligonucleotides are DNA or RNA molecules that are complementary to some portion of a mRNA molecule. When present in a cell, antisense oligonucleotides hybridize to an existing mRNA molecule and inhibit translation into a gene product. Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The methods of the invention include the use of antisense oligonucleotide to diminish the amount of a molecule that causes a decrease in the amount or activity PSAT1, thereby increasing the amount or activity of PSAT1. Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of a gene expressing a protein that diminishes the level or activity of PSAT1 can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28:4929; Altman et al., U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030), allowing the selective cleavage of specific mRNA molecules. Given the nucleotide sequence of the molecule, one of ordinary skill in the art could synthesize an antisense oligonucleotide or ribozyme without undue experimentation, provided with the disclosure and references incorporated herein.

### Polypeptides

In one embodiment, the composition of the present invention comprises a polypeptide activator of PSAT1. In one embodiment, the polypeptide directly enhances the activity of PSAT1. In one embodiment, the polypeptide inhibits the activity of a negative regulator of PSAT1 expression and/or activity. In one embodiment, the polypeptide comprises a PSAT1 polypeptide.

In one embodiment, the polypeptide comprises an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO: 3.

In one embodiment, the polypeptide comprises an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% of the length of SEQ ID NO: 3.

In one embodiment, the polypeptide comprises an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to an amino acid sequence that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% of the length of SEQ ID NO: 3.

In one embodiment, the polypeptide comprises the amino acid sequence of SEQ ID NO: 3.

The polypeptide of the present invention may be made using chemical methods. For example, polypeptides can be synthesized by solid phase techniques (Roberge J Y et al (1995) Science 269:202-204), cleaved from the resin, and purified by preparative high-performance liquid chromatography. Automated synthesis may be achieved, for example, using the ABI 431 A Peptide Synthesizer (Perkin Elmer) in accordance with the instructions provided by the manufacturer.

The invention should also be construed to include any form of a polypeptide having substantial homology to a polypeptide disclosed herein. In one embodiment, a polypeptide which is “substantially homologous” is about 50% homologous, about 70% homologous, about 80% homologous, about 90% homologous, about 95% homologous, or about 99% homologous to amino acid sequence of a polypeptide disclosed herein.

The polypeptide may alternatively be made by recombinant means or by cleavage from a longer polypeptide. The composition of a fusion protein may be confirmed by amino acid analysis or sequencing.

The variants of the polypeptide according to the present invention may be (i) one in which one or more of the amino acid residues are substituted with a conserved or non-conserved amino acid residue and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the polypeptide of the present invention, (iv) fragments of the polypeptide and/or (v) one in which the polypeptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two polypeptide is determined by comparing the amino acid sequence and its conserved amino acid substitutes of one polypeptide to a sequence of a second polypeptide. Variants are defined to include peptide sequences different from the original sequence. In one embodiment, variants are different from the original sequence in less than 40% of residues per segment of interest different from the original sequence in less than 25% of residues per segment of interest, different by less than 10% of residues per segment of interest, or different from the original protein sequence in just a few residues per segment of interest and at the same time sufficiently homologous to the original sequence to preserve the functionality of the original sequence and/or the ability to promote CM proliferation. The present invention includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences may be determined by using the BLASTP algorithm [BLAST Manual, Altschul, S., et al., NCBI NLM NIH Bethesda, Md. 20894, Altschul, S., et al., J. Mol. Biol. 215:403-410 (1990)].

The polypeptide of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The polypeptide of the invention may include unnatural amino acids formed by post-translational modification or by introducing unnatural amino acids during translation. A variety of approaches are available for introducing unnatural amino acids during protein translation.

A polypeptide of the invention may be phosphorylated using conventional methods such as the method described in Reedijk et al. (The EMBO Journal 11 (4): 1365, 1992).

Cyclic derivatives of the polypeptide of the invention are also part of the present invention. Cyclization may allow the polypeptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467). The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic fusion protein which is more flexible than the cyclic peptides containing peptide bond linkages as described above. A more flexible peptide may be prepared by introducing cysteines at the right and left position of the peptide and forming a disulfide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

A polypeptide of the invention may be synthesized by conventional techniques. For example, the polypeptide may be synthesized by chemical synthesis using solid phase peptide synthesis. These methods employ either solid or solution phase synthesis methods (see for example, J. M. Stewart, and J. D. Young, Solid Phase Peptide Synthesis, 2nd Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis). By way of example, a peptide of the invention may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a polypeptide of the invention conjugated with other molecules may be prepared by fusing, through recombinant techniques, the N-terminal or C-terminal of the polypeptide, and the sequence of a selected protein or selectable marker with a desired biological function. The resultant fusion proteins contain the polypeptide fused to the selected protein or marker protein as described herein. Examples of proteins which may be used to prepare fusion proteins include immunoglobulins, glutathione-S-transferase (GST), hemagglutinin (HA), and truncated myc.

Polypeptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The polypeptide of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

### Nucleic Acids

In one embodiment, the composition of the present invention comprises a nucleic acid molecule encoding a polypeptide activator of PSAT1. In one embodiment, the nucleic acid molecule encodes a polypeptide that directly enhances the activity of PSAT1. In one embodiment, the nucleic acid molecule encodes a polypeptide that inhibits the activity of a negative regulator of PSAT1 expression and/or activity. In one embodiment, the nucleic acid molecule inhibits the transcription and/or translation of a negative regulator of PSAT1 expression and/or activity. In one embodiment, the nucleic acid molecule encodes a PSAT1 polypeptide.

The nucleic acid molecule of the present invention may comprise any type of nucleic acid, including, but not limited to DNA and RNA. For example, in one embodiment, the composition comprises an isolated DNA molecule, including for example, an isolated cDNA molecule, encoding a polypeptide of the invention. In one embodiment, the composition comprises an isolated RNA molecule encoding a polypeptide of the invention, or a functional fragment thereof.

In one embodiment, the nucleic acid molecule comprises a DNA molecule. In one embodiment, the DNA molecule comprises a nucleotide sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO: 1.

In one embodiment, the DNA molecule comprises a nucleotide sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% of the length of SEQ ID NO: 1.

In one embodiment, the DNA molecule comprises a nucleotide sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to a nucleotide sequence that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% of the length of SEQ ID NO: 1.

In one embodiment, the DNA molecule comprises the nucleotide sequence of SEQ ID NO: 1.

In one embodiment, the nucleic acid molecule comprises an RNA molecule. In one embodiment, the RNA molecule comprises a nucleotide sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO: 2.

In one embodiment, the RNA molecule comprises a nucleotide sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% of the length of SEQ ID NO: 2.

In one embodiment, the RNA molecule comprises a nucleotide sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to a nucleotide sequence that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% of the length of SEQ ID NO: 2.

In one embodiment, the RNA molecule comprises the nucleotide sequence of SEQ ID NO: 2.

The nucleic acid molecule encoding a polypeptide of the invention can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Nucleic acid molecules discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, or as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, or different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone.

Modifications of the nucleic acid of the invention may be present at one or more of, a phosphate group, a sugar group, backbone, N-terminus, C-terminus, or nucleobase.

The nucleic acid molecules of the present invention can be modified to improve stability in serum or in growth medium for cell cultures. Modifications can be added to enhance expression, stability, functionality, and/or specificity and to minimize immunostimulatory properties of the nucleic acid molecule of the invention. For example, in order to enhance the stability, 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule. In one embodiment, the nucleic acid molecule is codon optimized for expression in mammalian cells.

In one embodiment of the present invention the nucleic acid molecule may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH2, NHR, NR2 or ON, wherein R is C1-C6 alkyl, alkenyl or alkynyl and halo is F, Cl, Br or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino) propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable.

In one embodiment, the nucleic acid molecule comprises methylpseudouridine in place of naturally occurring uridine to improve expression and reduce immunogenicity as compared to an endogenous RNA molecule. It should be noted that the above modifications may be combined.

In some instances, the nucleic acid molecule comprises at least one of the following chemical modifications: 2′-H, 2′-O-methyl, or 2′-OH modification of one or more nucleotides. In certain embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

The present invention also includes a vector in which the nucleic acid molecule of the present invention is inserted. The art is replete with suitable vectors that are useful in the present invention.

In brief summary, the expression of natural or synthetic nucleic acid molecules encoding a polypeptide of the invention is typically achieved by operably linking a nucleic acid sequence encoding the polypeptide of the invention or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. (See, e.g., U.S. Pat. Nos. 5,399,346, 5,580,859, 5,589,466, incorporated by reference herein in their entireties). In another embodiment, the invention provides a gene therapy vector.

The nucleic acid molecule of the invention can be cloned into a number of types of vectors. For example, the nucleic acid can be cloned into a vector including, but not limited to a plasmid, a phagemid, a phage derivative, an animal virus, and a cosmid. Vectors further include expression vectors, replication vectors, probe generation vectors, and sequencing vectors.

Additionally, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

RNA molecules of the present disclosure, such as a mRNA (e.g., modified mRNA) may be transcribed in vitro from template DNA, referred to as an “in vitro transcription template.” The source of the DNA can be, for example, genomic DNA, plasmid DNA, phage DNA, cDNA, synthetic DNA sequence or any other appropriate source of DNA. In some embodiments, an in vitro transcription template encodes a 5′ untranslated (UTR) region, contains an open reading frame, and encodes a 3′ UTR and a polyA tail. The particular nucleotide sequence composition and length of an in vitro transcription template will depend on the mRNA encoded by the template.

In one embodiment, 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, 5′ UTR can contain the Kozak sequence of the endogenous gene. Alternatively, when a 5′ UTR that is not endogenous to the gene of interest is being added by PCR as described above, a consensus Kozak sequence can be redesigned by adding 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments 5′ UTR can be derived from an RNA virus whose RNA genome is stable in cells. In other embodiments various nucleotide analogues can be used in 3′ or 5′ UTR to impede exonuclease degradation of the mRNA.

To enable synthesis of RNA from a DNA template, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 RNA polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the RNA molecule has both a cap on 5′ end and a 3′ poly(A) tail which determine ribosome binding, initiation of translation and stability of mRNA in the cell. On a circular DNA template, for instance, plasmid DNA, RNA polymerase produces a long concatameric product, which is not suitable for expression in eukaryotic cells. The transcription of plasmid DNA linearized at the end of 3′ UTR results in normal sized mRNA, which is effective in eukaryotic transfection when it is polyadenylated after transcription.

On a linear DNA template, phage T7 RNA polymerase can extend 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003)).

The conventional method of integration of polyA/T stretches into a DNA template is molecular cloning. However, polyA/T sequence integrated into plasmid DNA can cause plasmid instability, which can be ameliorated through the use of recombination incompetent bacterial cells for plasmid propagation.

Poly(A) tails of RNAs can be further extended following in vitro transcription with the use of a poly(A) polymerase, such as E. coli polyA polymerase (E-PAP) or yeast polyA polymerase. In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to mRNA molecules. In one embodiment, RNAs produced by the methods include a 5′ cap1 structure. Such cap1 structure can be generated using Vaccinia capping enzyme and 2′-O-methyltransferase enzymes (CellScript, Madison, WI). Alternatively, 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

### Delivery Vehicles

In various embodiments, the present invention relates to a composition comprising a delivery vehicle comprising one or more activators, polypeptides, or nucleic acids of the present. Exemplary delivery vehicles include, but are not limited to, microspheres, microparticles, nanoparticles, polymerosomes, liposomes, and micelles. For example, in some embodiments, the delivery vehicle is a lipid nanoparticle loaded with a nucleic acid of the present invention.

It should be recognized that any delivery vehicle known in the art as suitable for the delivery of one or more modulator of one or more activator, polypeptide, or nucleic acid of the present invention, can be used in the compositions and methods of the present invention.

The term “nanoparticle,” as used herein, refers to a particle having at least one dimension in the range of about 1 nm to about 1000 nm, including any integer value between 1 nm and 1000 nm (including about 1, 2, 5, 10, 20, 50, 60, 70, 80, 90, 100, 200, 500, 1000 nm, and all integers and fractional integers in between). In some embodiments, the nanoparticle has at least one dimension, e.g., a diameter, of about 100 nm. In other embodiments, the nanoparticle has a diameter of about 200 nm, a diameter of about 500 nm, or a diameter of about 1000 nm (1 μm). Nanoparticles having a diameter of at least 1000 nm also may be referred to as a “microparticle.” Thus, the term “microparticle” includes particles having at least one dimension in the range of about one micrometer (μm), i.e., 1×10⁻⁶ meters, to about 1000 μm. The term “particle” as used herein is meant to include nanoparticles and microparticles.

Nanoparticles suitable for use in the presently disclosed compositions and methods may exist in a variety of shapes, including, but not limited to, spheroids, rods, disks, pyramids, cubes, cylinders, nanohelixes, nanosprings, nanorings, rod-shaped nanoparticles, arrow-shaped nanoparticles, teardrop-shaped nanoparticles, tetrapod-shaped nanoparticles, prism-shaped nanoparticles, and a plurality of other geometric and non-geometric shapes. In some embodiments, the disclosed nanoparticles have a spherical shape.

The delivery vehicles of the invention can be formulated for administration in a pharmaceutical carrier in accordance with known techniques. See, e.g., Remington: The Science and Practice of Pharmacy, 21st Edition (2005), Lippincott Williams & Wilkins, Philadelphia, Pa.). In the manufacture of a pharmaceutical formulation according to the invention, the compound (including the physiologically acceptable salts thereof) is typically admixed with, inter alia, an acceptable carrier. The carrier can be a solid or a liquid, or both. In one embodiment, the carrier is formulated with the compound as a unit-dose formulation, for example, a tablet, which can contain from 0.01 or 0.5% to 95% or 99% by weight of the compound. One or more compounds can be incorporated in the formulations of the invention, which can be prepared by any of the well-known techniques of pharmacy.

In some embodiments, a delivery vehicle comprises one or more selected from the group consisting of: a polyalkylene glycol, a polycation and a targeting moiety. In one embodiment, the nanoparticle comprises a polyalkylene glycol, a polycation and a targeting moiety.

“Polyalkylene glycol” means straight or branched polyalkylene glycol polymers including, but not limited to, polyethylene glycol (PEG), polypropylene glycol (PPG), and polybutylene glycol (PBG), as well as co-polymers of PEG, PPG and PBG in any combination, and includes the monoalkylether of the polyalkylene glycol. Thus, in various embodiments of this invention, the polyalkylene glycol in the nanoparticles of this invention can be, but is not limited to, polyethylene glycol, polypropylene glycol, polybutylene glycol, and any combination thereof.

In certain embodiments, the polyalkylene glycol of the delivery vehicle is polyethylene glycol or “PEG.” The term “PEG subunit” refers to a single polyethylene glycol unit, i.e., —(CH2CH2O)—.

PEG of any suitable molecular weight may be employed in the nanoparticle, and is available over a wide range of molecular weights. The PEG molecular weight may be, for example, between about 300 g/mol to about 10,000,000 g/mol (e.g., about 600, 1,000, 5,000, 10,000 g/mol, or a range defined by any two of the foregoing values).

In some embodiments, the delivery vehicle of the present invention comprises a polycation. The term “polycation” refers to a compound having a positive charge, for example, at least 2 positive charges, at a selected pH, for example, at physiological pH. Polycationic moieties have between about 2 to about 15 positive charges, for example, between about 2 to about 12 positive charges. In one embodiment, the polycation has between about 2 to about 8 positive charges at selected pH values.

Many polycations are known in the art. Suitable constituents of polycations include basic amino acids and their derivatives such as arginine, asparagine, glutamine, lysine and histidine; cationic dendrimers; and amino polysaccharides. Suitable polycations can be linear, such as linear tetralysine, branched or dendrimeric in structure.

In some embodiments of the invention, the polycation can be, but is not limited to polyethyleneimine, polyethylenimine, poly(allylanion hydrochloride; PAH), putrescine, cadaverine, polylysine, poly-arginine, poly(trimethylenimine), poly(tetramethylenimine), polypropylenimine, aminoglycoside-polyamine, dideoxy-diamino-b-cyclodextrin, spermine, spermidine, cadaverine, poly(2-dimethylamino)ethyl methacrylate, poly(histidine), cationized gelatin, dendrimers, chitosan, and any combination thereof. Exemplary polycations also include, but are not limited to, synthetic polycations based on acrylamide and 2-acrylamido-2-methylpropanetrimethylamine, poly(N-ethyl-4-vinylpyridine) or similar quarternized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, and polypeptides such as protamine, the histone polypeptides, polylysine, polyarginine and polyomithine.

In certain embodiments of the delivery vehicle of this invention, the polycation is polyethylenimine (PEI). The amine-rich cationic polymer polyethylenimine (PEI) is an efficient nucleotide carrier that binds to the negatively-charged phosphate backbone of nucleotides and negatively-charged elements of cell membranes, facilitating endocytotic uptake of PEI-nucleotide complexes into cells (Bieber, T. et. al., 2002, Journal of Controlled Release, 82:441-454; Boussif, O., et. al., 1995, Proceedings of the National Academy of Sciences U.S.A., 92:7297-7301; Godbey, W. T., et. al., 1999, Proceedings of the National Academy of Sciences U.S.A., 96:5177-5181; Kichler, A., 2004, Journal of Gene Medicine, 6 (Supplement 1): S3-10; Petersen, H., et. al., 2002, Bioconjugate Chemistry, 13:845-854; Petersen, H., et. al., 2002, Macromolecules, 35:6867-6874; Suh, J., et. al., 2003, Proceedings of the National Academy of Sciences U.S.A., 100:3878-3882; Thomas, M., et. al., 2003, Applied Microbiology and Biotechnology, 62:27-34). Protonation of the amine groups on PEI within endosomal compartments (the “so called proton sponge effect) is thought to cause osmotic lysis, and release of the endosomal contents (Akinc, A., et. al., 2005, Journal of Gene Medicine, 7 (5): 657-663; Boussif, O., et. al., 1995, Proceedings of the National Academy of Sciences U.S.A., 92:7297-7301; Hara-Chikuma, M., et. al., 2004, Journal of Biological Chemistry, 280 (2): 1241-1247; Sonawane, N. D., et. al., 2003, Journal of Biological Chemistry, 278:44826-44831; Thomas, M., et. al., 2003, Applied Microbiology and Biotechnology, 62:27-34).

In various embodiments of this invention, the polycation (e.g., PEI) can be complexed with an active agent of this invention (e.g., a polynucleotide, an oligonucleotide, an anionic protein, an anionic drug, a polynucleotide or oligonucleotide covalently bonded to a peptide or protein, as well as any combination thereof) via physical electrostatic force (e.g., wherein the negative charges in the active agent(s) bind with the positive charges in the polycation.

In some embodiments, the delivery vehicle is functionalized for enhanced delivery to target tissues and enhanced cellular uptake of the one or more modulator of one or more chemoresistance promoting molecule, the one or more chemosensitivity promoting molecule, or a combination thereof. Addition of functional groups to the nanoparticle can improve stability, improve biodistribution, improve tissue delivery of the cargo (i.e. the one or more inhibitor of the one or more chemoresistance promoting molecule), improve nanoparticle and/or cellular uptake, and any combinations thereof. Non-limiting examples of functional groups are gold nanoparticles (GNP), TAT-PTD and derivatives thereof, ApoE, albumin, antibody, antibody fragment, magnetic nanoparticle, iron oxide, transferrin, AAV tropism fragment, and combinations thereof. In some embodiments, the functional group is a targeting moiety.

### Pharmaceutical Compositions and Formulations

The present invention also encompasses the use of pharmaceutical compositions of the invention or salts thereof to practice the methods of the invention. Such a pharmaceutical composition may consist of at least one composition of the invention (e.g., an activator, polypeptide, nucleic acid molecule, delivery vehicle, etc.) or a salt thereof in a form suitable for administration to a subject, or the pharmaceutical composition may comprise at least one composition of the invention (e.g., an activator, polypeptide, nucleic acid molecule, delivery vehicle, etc.) or a salt thereof, and one or more pharmaceutically acceptable carriers, one or more additional ingredients, or some combination of these. The compound of the invention may be present in the pharmaceutical composition in the form of a physiologically acceptable salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol are included in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, include a mammal, for example a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments there between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. An exemplary preservative is a combination of about 0.5% to 2.0% benzyl alcohol and 0.05% to 0.5% sorbic acid.

In one embodiment, the composition includes an anti-oxidant and a chelating agent that inhibits the degradation of the compound. Exemplary antioxidants for some compounds are BHT, BHA, alpha-tocopherol and ascorbic acid in the range of about 0.01% to 0.3% and BHT in the range of 0.03% to 0.1% by weight by total weight of the composition. In one embodiment, the chelating agent is present in an amount of from 0.01% to 0.5% by weight by total weight of the composition. Exemplary chelating agents include edetate salts (e.g. disodium edetate) and citric acid in the weight range of about 0.01% to 0.20%. In some embodiments, the chelating agent is in the range of 0.02% to 0.10% by weight by total weight of the composition. The chelating agent is useful for chelating metal ions in the composition that may be detrimental to the shelf life of the formulation. While BHT and disodium edetate are exemplary antioxidants and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil in water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Methods

In various embodiments, the present invention comprises methods of administering to subject in need thereof, a composition that regenerates cardiac tissue. In some embodiments, the present invention comprises a method of regenerating damaged cardiac tissue in a subject in need thereof. In some embodiments, the present invention comprises a method of treating one or more disease or disorder associated with cardiac tissue damage in a subject in need thereof. In some embodiments, the method comprises administering a composition that promotes CM proliferation. In some embodiments, the method comprises administering a composition that inhibits one or more selected from the group consisting of: apoptosis, oxidative stress and DNA damage. In some embodiments, the method comprises administering one or more selected from the group consisting of: a PSAT1 activator, a PSAT1 polypeptide, a nucleic acid molecule encoding PSAT1, and a nanoparticle comprising a PSAT1 activator, PSAT1 polypeptide, or nucleic acid encoding PSAT1, as described above.

In some embodiments, the subject has experienced one or more adverse event resulting in cardiac tissue damage. In some embodiments, the adverse event resulting in cardiac tissue damage is myocardial infarction.

In one embodiment, the subject is a cell. In one embodiment, the subject is a mammal. For example, in one embodiment, the subject is a human, non-human primate, dog, cat, horse, cow, goat, sheep, rabbit, pig, rat, or mouse.

In some embodiments, the disease or disorder associated with cardiac tissue damage, includes but is not limited to, fibrotic diseases, myocardial infarction, ischemic heart disease, heart failure, and dilated cardiomyopathy (DCM).

In one embodiment, the methods described above comprise administering a pharmaceutical composition comprising one or more PSAT1 activator of the present invention. In an embodiment, the pharmaceutical compositions useful for practicing the methods of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

In some embodiments, the method comprises administering to the subject one or more additional therapies. Examples of additional therapies that can be administered include, but are not limited to, antifibrotic agents, anticlotting agents, clot-reducing treatments, oxygen therapy, vasodialators, anxiolytic agents, angioplasty, atherectomy, bypass surgery, pacemaker insertion, insertion of a left ventricular assist device (LVAD), anticoagulants, aspirin, beta blockers, statins, ACE inhibitors, calcium channel blockers, diuretics, nitrates, metabolic modulators, angiotensin-2 receptor blockers, neprilysin inhibitors, smooth muscle relaxants, and combinations thereof.

One of skill in the art will appreciate that a PSAT1 polypeptide, a recombinant PSAT1 polypeptide, or an active PSAT1 polypeptide fragment can be administered singly or in any combination thereof. Further, a PSAT1 polypeptide, a recombinant PSAT1 polypeptide, or an active PSAT1 polypeptide fragment can be administered singly or in any combination thereof in a temporal sense, in that they may be administered simultaneously, before, and/or after each other. One of ordinary skill in the art will appreciate, based on the disclosure provided herein, that a PSAT1 polypeptide, a recombinant PSAT1 polypeptide, or an active PSAT1 polypeptide fragment can be used to prevent or treat damaged cardiac tissue, and that an activator can be used alone or in any combination with another PSAT1 polypeptide, recombinant PSAT1 polypeptide, active PSAT1 polypeptide fragment, or PSAT1 activator to effect a therapeutic result.

One of skill in the art, when armed with the disclosure herein, would appreciate that the treating of damaged cardiac tissue encompasses administering to a subject a PSAT1 polypeptide, a recombinant PSAT1 polypeptide, an active PSAT1 polypeptide fragment, or PSAT1 activator as a preventative measure against a disease or disorder associated with damaged cardiac tissue. As more fully discussed elsewhere herein, methods of increasing the level or activity of a PSAT1 encompass a wide plethora of techniques for increasing not only PSAT1 activity, but also for increasing expression of a nucleic acid encoding PSAT1. Additionally, as disclosed elsewhere herein, one skilled in the art would understand, once armed with the teaching provided herein, that the present invention encompasses a method of preventing a wide variety of diseases where increased expression and/or activity of PSAT1 mediates, treats or prevents the resultant cardiac tissue damage. Further, the invention encompasses treatment or prevention of such diseases discovered in the future.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for oral, rectal, vaginal, parenteral, topical, pulmonary, intranasal, buccal, ophthalmic, or another route of administration. A composition useful within the methods of the invention may be directly administered to the skin, or any other tissue of a mammal. Other contemplated formulations include liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human subject being treated, and the like.

Routes of administration of any of the compositions of the invention include oral, nasal, parenteral, sublingual, transdermal, transmucosal (e.g., sublingual, lingual, (trans) buccal, and (intra) nasal), intravesical, intraduodenal, intragastrical, rectal, intra-peritoneal, subcutaneous, intramuscular, intradermal, intra-arterial, intravenous, or intra-myocardial administration. In some embodiments, the composition of the invention is administered via intra-myocardial injection. In some embodiments, said intra-myocardial injection is localized to the peri-infarct area of an infarcted heart in need of cardiac tissue regeneration.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: PSAT1 as a Novel Yap-Regulated Inducer of Cardiac Regeneration

In the present Example, it is shown that hiPSC-EV treatment augments cardiac functions and induces significant CM cell cycling in mice post-MI. Proteomic analysis of hiPSC-EVs identified PSAT1 (phosphoserine aminotransferase 1) as a protein expressed exclusively in hiPSC-EVs. The role of PSAT1 in CM proliferation and cardiac regeneration has not previously been studied. The cardiac delivery of PSAT1 modRNA induces a significant CM proliferation post-MI. This increase in the CM cell cycle by the PSAT1 modRNA was associated with reduced scar size, improved cardiac function, reduced oxidative stress, CM apoptosis, and survival 28 days post-MI. Furthermore, the PSAT1 modRNA post-MI inhibits CM apoptosis by reducing oxidative stress and the DNA damage response. Finally, it is shown herein that the Yap, a master regulator of CM proliferation and cardiac regeneration, binds to the promoter of PSAT1 and induces its expression. Moreover, PSAT1 modRNA induces the serine biosynthesis pathway in CMs resulting in increased nucleotide synthesis and reduced oxidative stress, thereby supporting CM proliferation. In summary, the studies outlined herein uncover a novel role of YAP-regulated PSAT1 in post-MI CM proliferation and cardiac function. Furthermore, while not being bound by scientific theory, these studies demonstrate that a PSAT1 modRNA approach to enhancing CM proliferation and cardiac function in mouse models may be adapted for mRNA therapeutics in a clinical setting.

In the present Example a modRNA approach was used to express PSAT1 in the heart post-MI transiently (Magadum, A., et al., 2020, Circulation, 141:1249-1265; Magadum, A., et al., 2018, Molecular Therapy-Nucleic Acids, 13:133-143; Magadum, A., et al., 2019, Molecular Therapy, 27:785-793; Sultana, N., et al., 2017, Molecular Therapy, 25:1306-1315; Zangi, L., eta 1., 2013, Nature Biotechnology, 898-907; Magadum, A., et al., 2020, Circulation142: 2485-2488; Hadas, Y., et al., 2020, Circulation, 141:916-930). As was shown previously, ModRNA transfection induces transient, pulse-like protein expression in vitro (rat neonatal CMs) as well as in vivo (mouse adult heart) (7-12 days) (Magadum, A., et al., 2020, Circulation, 141:1249-1265; Magadum, A., et al., 2018, Molecular Therapy-Nucleic Acids, 13:133-143; Magadum, A., et al., 2019, Molecular Therapy, 27:785-793; Sultana, N., et al., 2017, Molecular Therapy, 25:1306-1315; Zangi, L., eta 1., 2013, Nature Biotechnology, 898-907; Magadum, A., et al., 2020, Circulation 142: 2485-2488; Hadas, Y., et al., 2020, Circulation, 141:916-930). Induced pluripotent stem cells (iPSC) derivatives have been used in preclinical models of cell replacement (Lalit, P. A., et al., 2014, Circulation Research, 114:1328-1345; Gao, L., et al., 2020, Science Translational Medicine, 12 (561): eaay 1318). It is well known that stem cell (mouse embryonic or iPSC)-derived exosomes induce cardiac protective role and improve cardiac function post-MI (Adamiak, M., et al., 2018, Circulation Research, 122:296-309; Garikipati, V. N. S., et al., 2018, Circulation Research, 123:188-204). While the role of mouse embryonic-derived EVs in CM proliferation is known, little is known about the CM proliferative ability of hiPSC-EVs (Khan, M., et al., 2015, Circulation Research, 117:52-64).

PSAT1 is Enriched in hiPSC-Derived EVs and Minimally Expressed in Adult Hearts

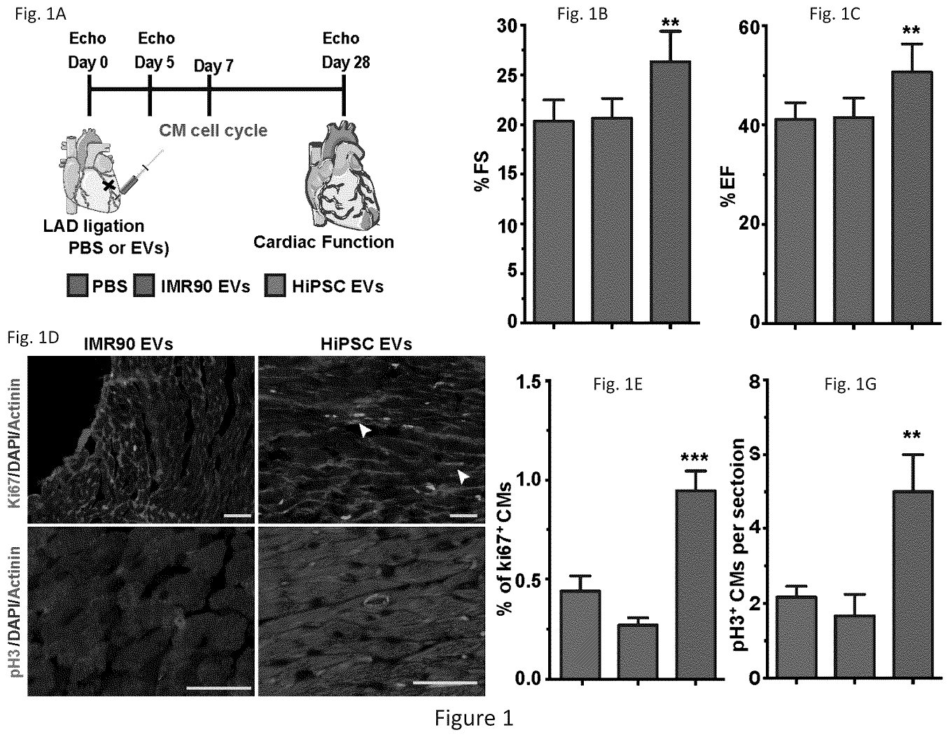

Induced pluripotent stem cells (iPSC) derivatives have been used in the preclinical model of cell replacement (Lalit, P. A., et al., 2014, Circulation Research, 114:1328-1345; Gao, L., et al., 2020, Science Translational Medicine, 12 (561): eaay 1318). It is well known that stem cell (mouse embryonic or iPSC) derived extracellular vesicles (EV) induce cardiac protective role and improve cardiac function post-myocardial infarction (post-MI) (Adamiak, M., et al., 2018, Circulation Research, 122:296-309; Garikipati, V. N. S., et al., 2018, Circulation Research, 123:188-204). While mouse embryonic stem cell-derived EVs role in CM proliferation is known, but not much is known about the CM proliferative ability of hiPSC-EVs (Khan, M., et al., 2015, Circulation Research, 117:52-64). Therefore, it was first determined whether hiPSC-EV-mediated improvements in cardiac function post-MI involves enhanced CM cell cycle and proliferation. hiPSC-EVs, control IMR90 fibroblast-EVs or PBS was injected in the infarct border zone of murine hearts post-MI and cardiac functions were analyzed after 28 days (FIG. 1A). It was found that hiPSC-EV-injected mice had improved ejection fraction (% EF) as well as % of fractional shortening (% FS) compared to PBS or IMR90-EVs post-MI (FIGS. 1B and 1C). It was further found that hiPSC-EVs significantly induced CM cell cycling (ki67+) and mitosis (pH3+), in mouse hearts compared to IMR90-EVs or PBS injected mice, seven days-post-MI (DPMI) (FIGS. 1D-1F).

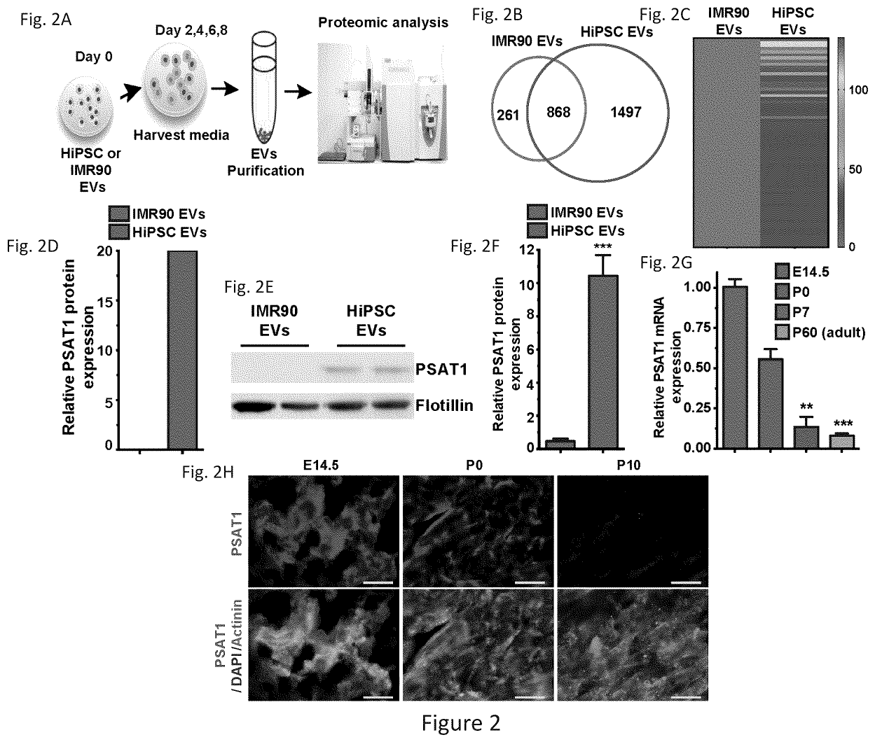

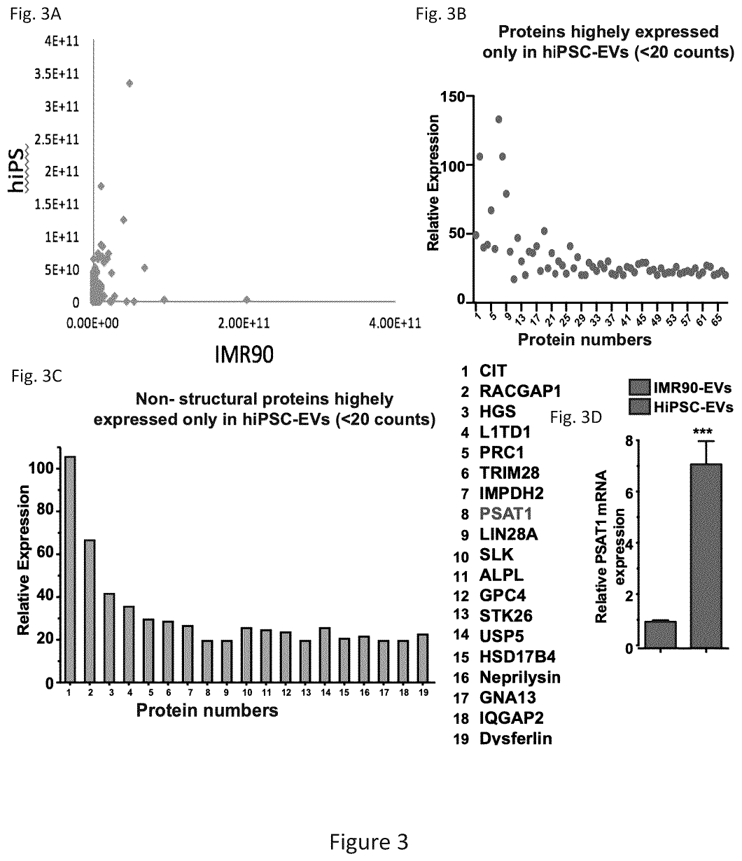

The role of hiPSC-EV derived miRNA and mRNAs in cardiac biology is studied, but very little is known about EV proteins and their function. Therefore, first, a protein expression analysis of hiPSC, or IMR-90 derived EVs was analyzed using mass spectrometry (FIG. 2A). This proteomic study provided multiple differential regulated proteins between the hiPSC, or IMR90 derived EVs, and some proteins were expressed exclusively in hiPSC-EVs (FIGS. 2A-2C and 3A). Non-structural proteins exclusively expressed in hiPSC-EVs were specifically analyzed, and 19 were found (FIGS. 3B and 3C). Using this data, combined with a developmentally restricted gene database of mouse hearts, PSAT1 was found to be a hiPSC-EV specific protein highly expressed in hiPSC-EVs, highly abundant in embryonic development but quickly downregulated after the postnatal period in mice hearts, with minimal expression in adult hearts (FIGS. 2D and 2G). PSAT1 expression in hiPSC-EVs was validated at both protein and mRNA levels (FIGS. 2E, 2F, and 3D).

PSAT1 modRNA Induces CM Proliferation Post-MI

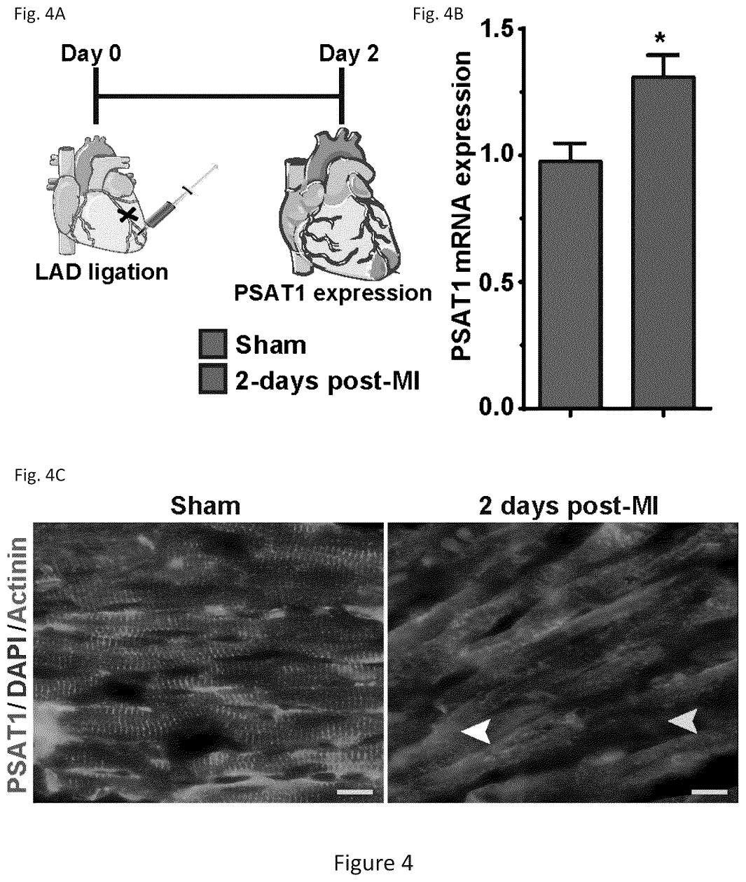

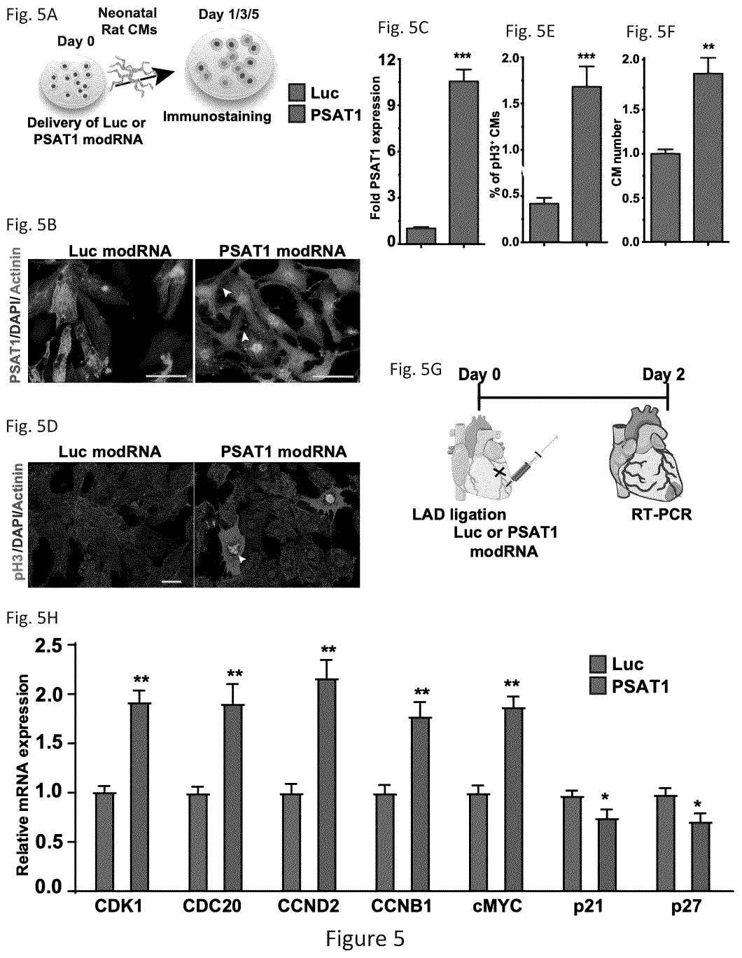

It was observed that post-MI PSAT1 expression in the heart increases moderately, however this is largely in non-CMs (FIG. 4). To study whether exogenous delivery PSAT1 in NRVM (neonatal rat ventricular CMs) or adult CMs post-MI induces the CM cell cycle, a modRNA delivery approach was used to express PSAT1 in vitro and in the post-MI hearts (Magadum, A., et al., 2020, Circulation, 141:1249-1265; Sultana, N., et al., 2017, Molecular Therapy, 25:1306-1315; Magadum, A., et al., 2018, Molecular Therapy-Nucleic Acids, 13:133-143; Zangi, L., et al., 2013, Nature Biotechnology, 31:898-907; Magadum, A., et al., 2019, Molecular Therapy, 27:785-793; Magadum, A., et al., 2020, Circulation, 142:2485-2488; Hadas, Y., et al., 2020, Circulation, 141:916-930). PSAT1 modRNA was designed and chemically synthesized in vitro. PSAT1 protein expression in NRVM was achieved within hours of administration and lasted up to several days (FIG. 5A-5C). Next, the effect of PSAT1 modRNA on the CM cell cycle was determined after 3 days, and it was found that PSAT1 modRNA significantly induced CM mitosis (pH3⁺) and increased CM number in 5 days (FIGS. 5A and 5D-5F).

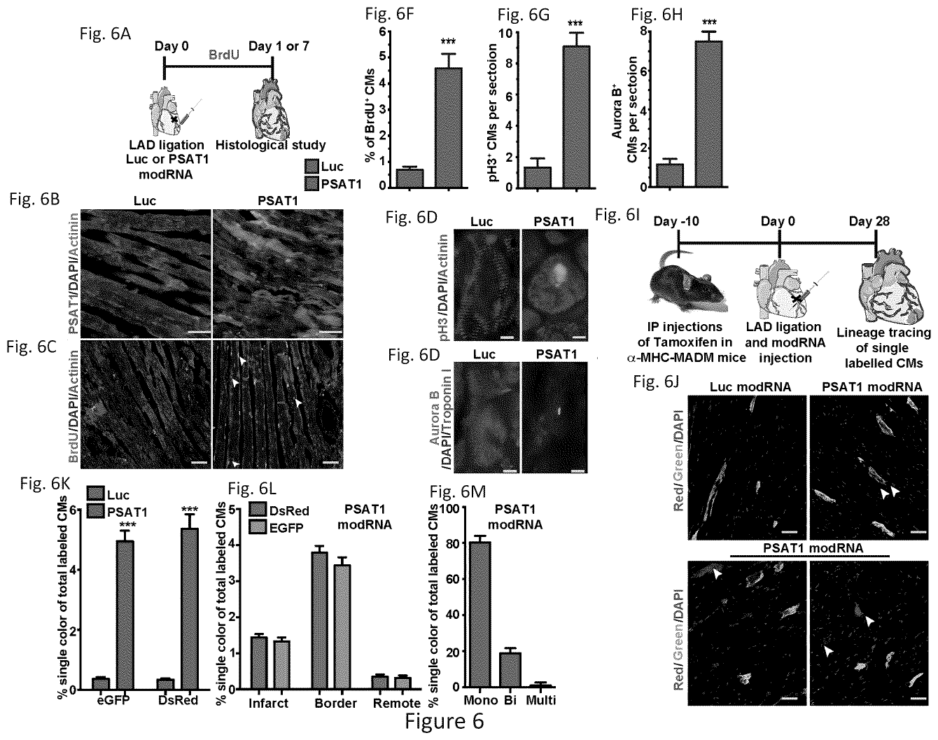

Increased dedifferentiation of CMs post-PSAT1 modRNA expression was observed, suggesting CMs undergoing mitosis. To see whether the PSAT1 modRNA can induce the CM cell cycle in the adult mice after MI, 100 μg of PSAT1 modRNA was delivered into mouse myocardium post-MI and robust PSAT1 protein expression was observed within 24 hours (FIGS. 6A and 6B). The immuno-staining analysis showed PSAT1 modRNA induced multiple markers of CM cell cycle and proliferation, including significant increase in CMs positive for BrdU (DNA synthesis), pH3, and Aurora B, at seven days post-MI compared to Luc modRNA (FIGS. 6C-6H). It was also found that PSAT1 modRNA induced the expression of positive cell cycle markers while inhibiting negative regulators of cell cycle markers compared to Luc modRNA (FIGS. 5G and 5H). Furthermore, CM-specific MADM mice (α-MHC-MADM mice; lineage-tracing model based on Cre-recombinase dependent mosaic analysis with double markers (MADM) mice) were used to analyze whether PSAT1 induces true CM cell division post-MI. Myocardial delivery of PSAT1 modRNA increased the % single color (green or red positive) compared to the total labeled CMs significantly, mainly in the infarct border zone, 1-month post-MI compared to Luc modRNA (FIGS. 6I-6L). It was also found that the majority of the single color (green or red positive) CMs of total labeled CMs were mononucleated (FIG. 6M).

PSAT1 modRNA Improves Cardiac Function Post-MI

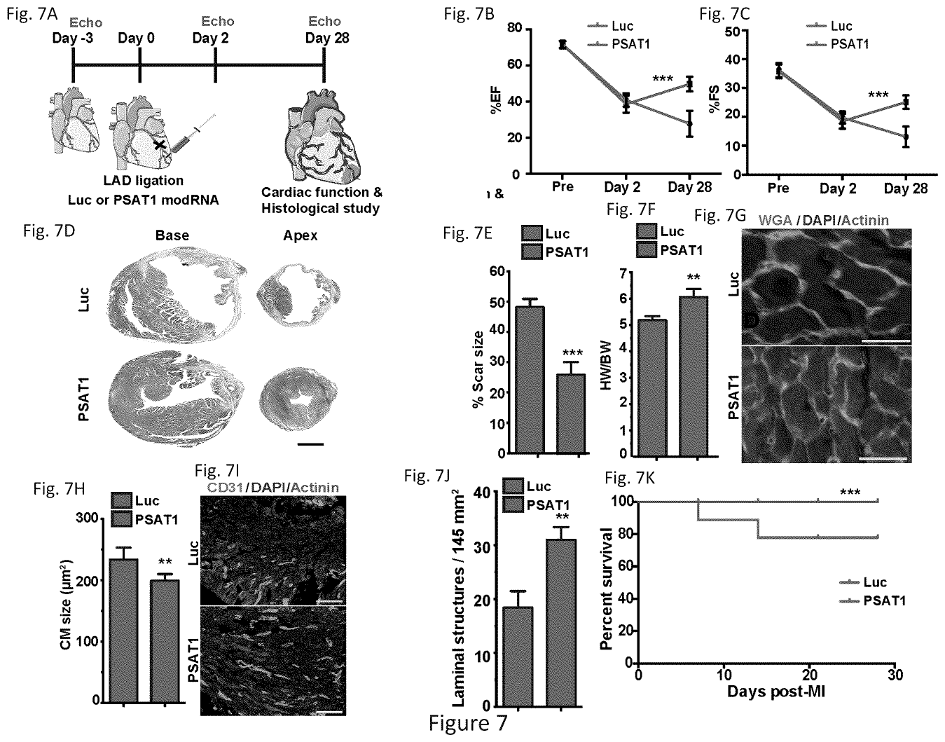

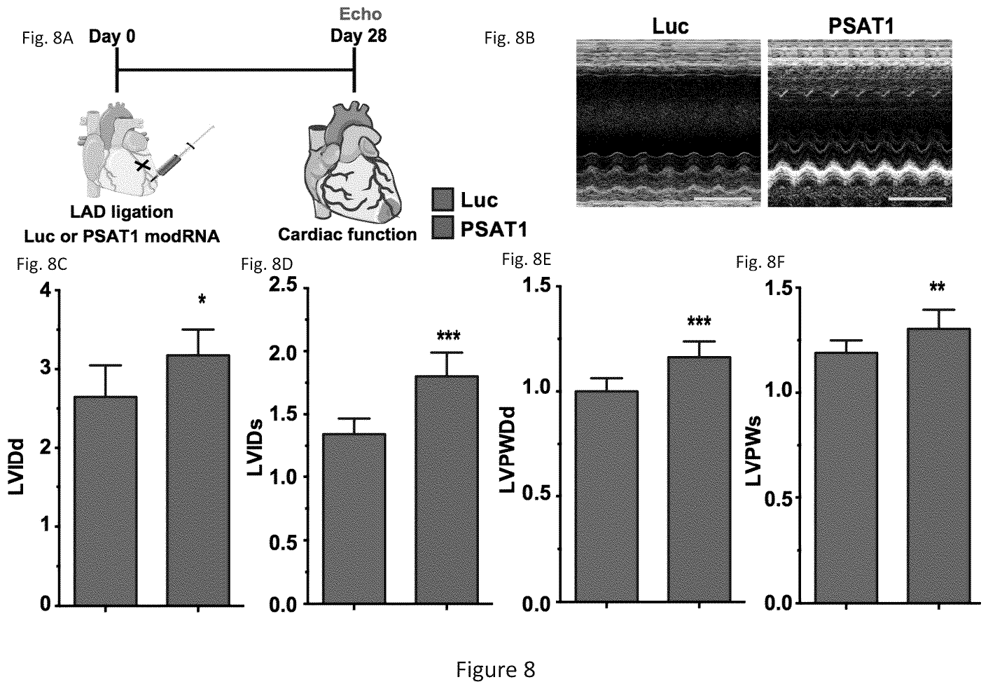

To study the impact of increased CM cell cycle on cardiac remodeling and improve cardiac function post-MI, the cardiac function and structure 28 days after PSAT1 modRNA delivery was also analyzed (FIG. 7A). Echocardiography studies showed that the prompt myocardial delivery of PSAT1 modRNA significantly improved the Ejection Fraction (% EF) and Fractional Shortening (% FS) post-MI compared to Luc (FIGS. 7B and 7C). Improvements in LVIDd and LVIDs (Left ventricular internal diameter end-diastole and end-systole) were observed post-MI and PSAT1 expression compared to control. Further, PSAT1 modRNA expression significantly increased LV end-diastolic or systolic posterior wall thickness (FIGS. 8A-8F). Masson's trichrome staining of mouse heart showed decreased scar size in PSAT1 modRNA injected mice compared to Luc modRNA (FIGS. 7D and 7E). The heart weight to body weight (HW/BW) ratio was also significantly increased (FIG. 7F). At the same time, the CM size significantly decreased in PSAT1 modRNA injected mice (FIGS. 7G and 7H), suggesting increased new muscle formation without a significant increase in cardiac hypertrophy. Finally, it was observed that PSAT1 modRNA also improved capillary density post-MI (FIGS. 7I and 7J). As a result of improved cardiac function, reduced scar size, induced CM proliferation, and angiogenesis, mice survival in PSAT1 injected mice was also significantly improved (FIG. 7K). Taken together, the present data confirm that PSAT1 expression induces CM proliferation, cardiac function, angiogenesis while the reduction in the scar size, suggesting a reversal of cardiac remodeling post-MI.

PSAT1 modRNA Activates Serine and Nucleotide Pathways

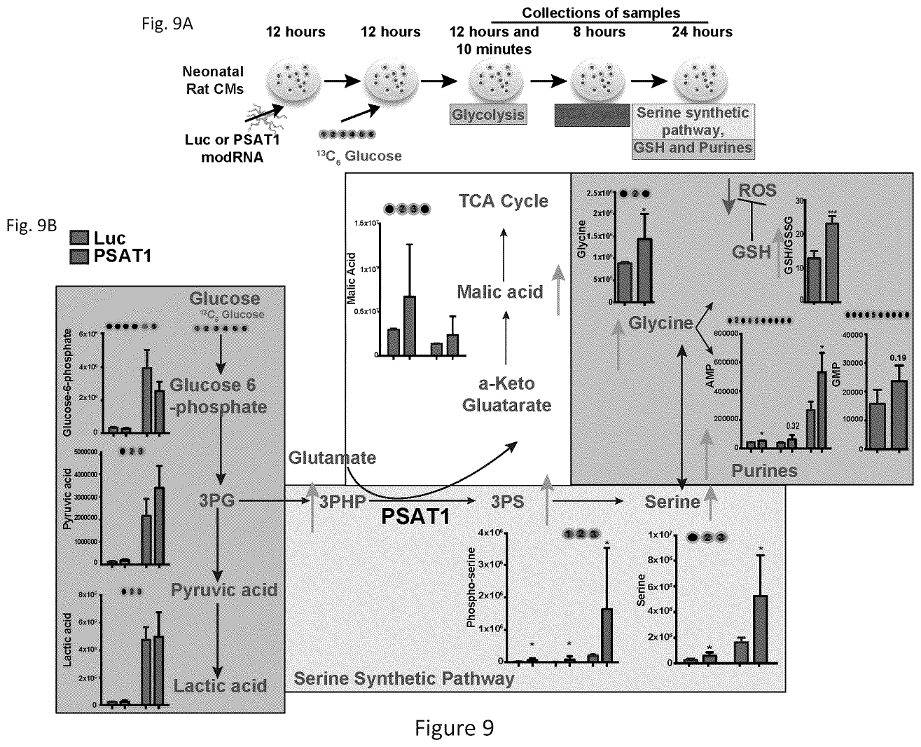

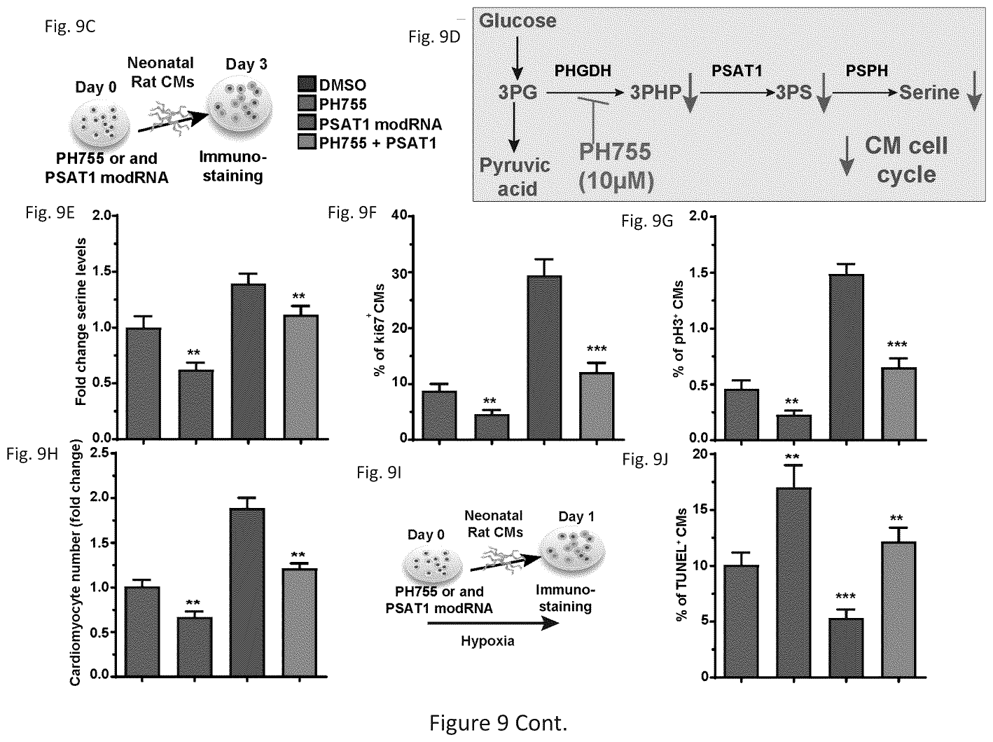

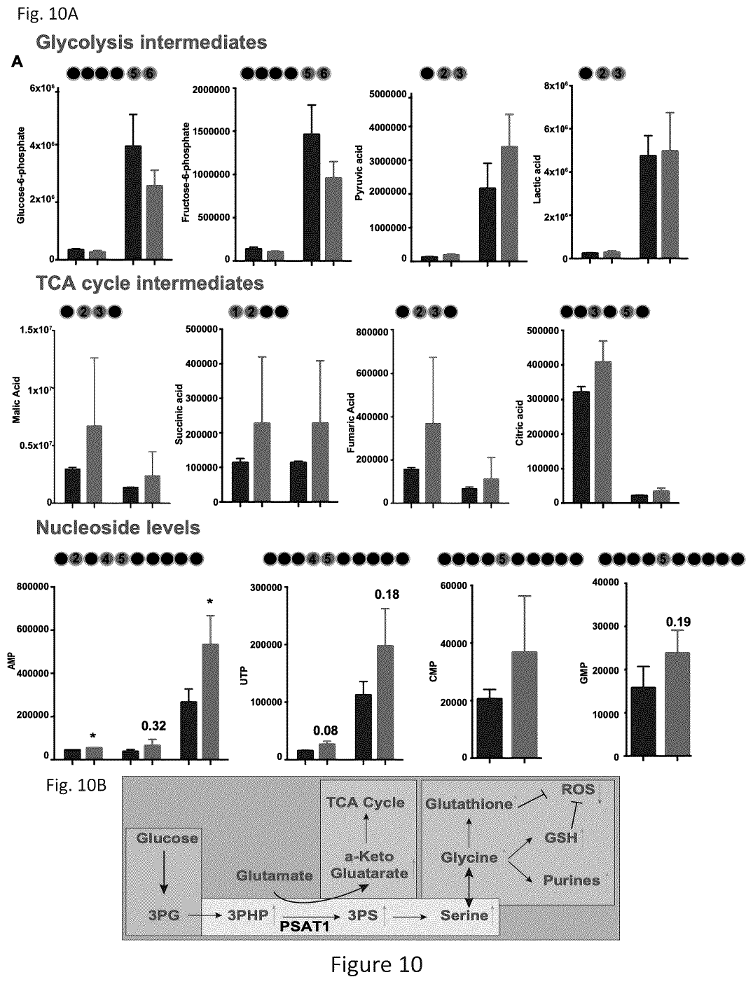

PSAT1 is an enzyme involved in the serine (amino acid) metabolic pathway where it converts 3-phosphohydroxy-pyruvate (3PHP), a glycolysis intermediate, and glutamate into phosphor-serine (3PS) and alpha keto-glutarate, which goes to TCA cycle. The 3PS synthesized is further enzymatically converted into the serine. To analyze whether serine synthesis increased under PSAT1 modRNA overexpression, ¹³C isotopic tracers for metabolic flux analysis were performed by using ¹³C glucose supplemented media in neonatal rat CMs 12 hours post-delivery of PSAT1 or Luc modRNA and collecting samples for analysis of glycolysis (10 minutes), TCA cycle (6 hours) or PPP (24 hours) after introducing media with the ¹³C glucose (FIGS. 9A and 9B). The results show a significant elevation of the metabolites involved in the serine synthesis pathway (SSP), including phosphoserine, serine, and glycine, suggesting an increased serine biosynthetic pathway (FIG. 9B). Increased levels of TCA cycle intermediates suggest that alpha-ketoglutarate synthesized after PSAT1 modRNA treatment entered the TCA cycle (FIGS. 10A and 10B).

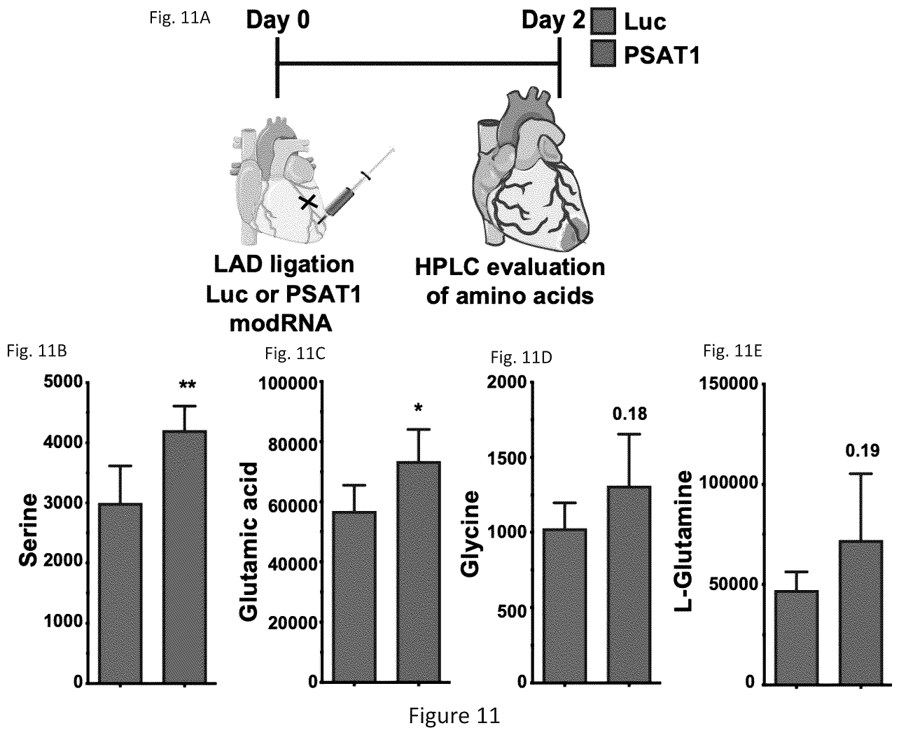

Furthermore, high glycine levels are known as precursors for glutathione (which is known to inhibit ROS production) and purine nucleotide synthesis (Ruiz-Ramirez, A., et al., 2014, Clinical Science, 126:19-29). PSAT1 modRNA treatment post-MI shows an increase in the levels of serine and glycine (HPLC study), which correlate with NRVM ¹³C glucose flux studies (FIGS. 11A-11E). Increased glutathione levels and increased purines AMP and GMP levels were observed after PSAT1 modRNA treatment, suggesting these increased purines likely provide the raw material for DNA synthesis in post-natal CMs (FIGS. 9B and 10B).

Next, it was examined if inhibition of SSP has an effect on CM proliferation and apoptosis. PH775, an inhibitor of PHGDH (the first rate-limiting step in SSP), was used in vitro at a concentration of 10 μM. PH755 treatment significantly impeded the CM cell cycle (Ki67⁺ or pH3⁺ CMs) and the CM number at the basal level (FIGS. 9E-9H). At the same time, PSAT1 modRNA expression under SSP inhibition partially reversed the CM cell cycle restriction compared to the control, but at a significantly lower rate than PSAT1 modRNA alone (FIGS. 9E-9H). Furthermore, PH755 treatment significantly increased the NRVM apoptosis under hypoxia. While PSAT1 modRNA alone significantly inhibited CM apoptosis under hypoxic conditions, PH755 treatment inhibits this (FIGS. 9I and 9J). Overall, the data suggest PSAT1 induces SSP in CMs and provides the nucleotides required for DNA synthesis. A higher level of GSH hinders oxidative stress and CM apoptosis. In contrast, inhibition of SSP impedes CM proliferation and promotes CM apoptosis, establishing SSP as a regulator of CM proliferation and apoptosis.

PSAT1 modRNA Inhibits Apoptosis, Oxidative Stress, and DNA Damage Response Post-MI

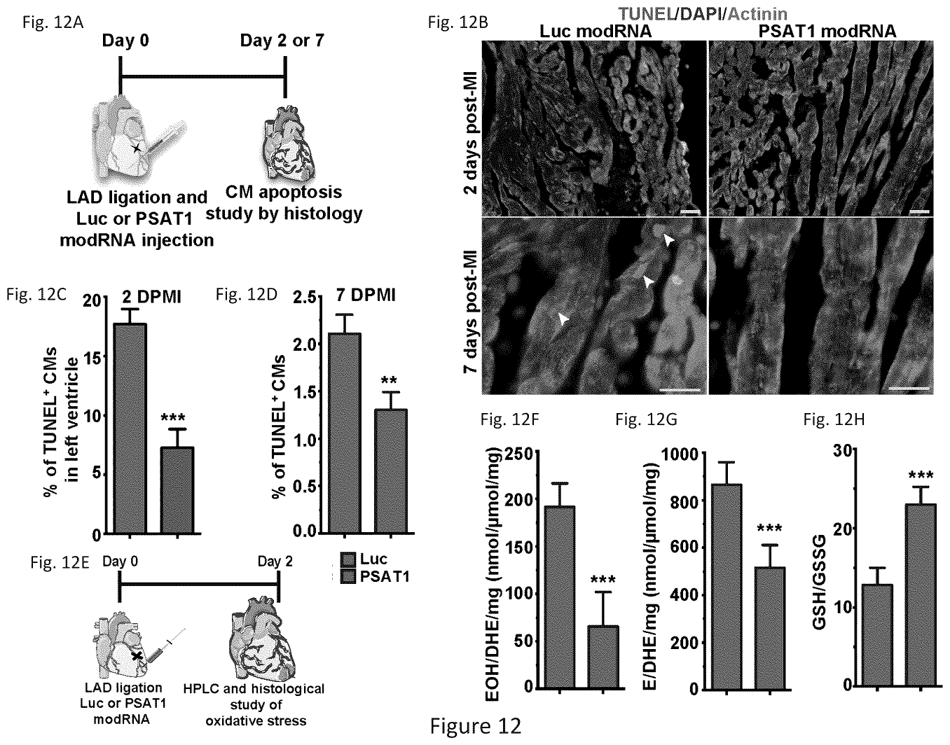

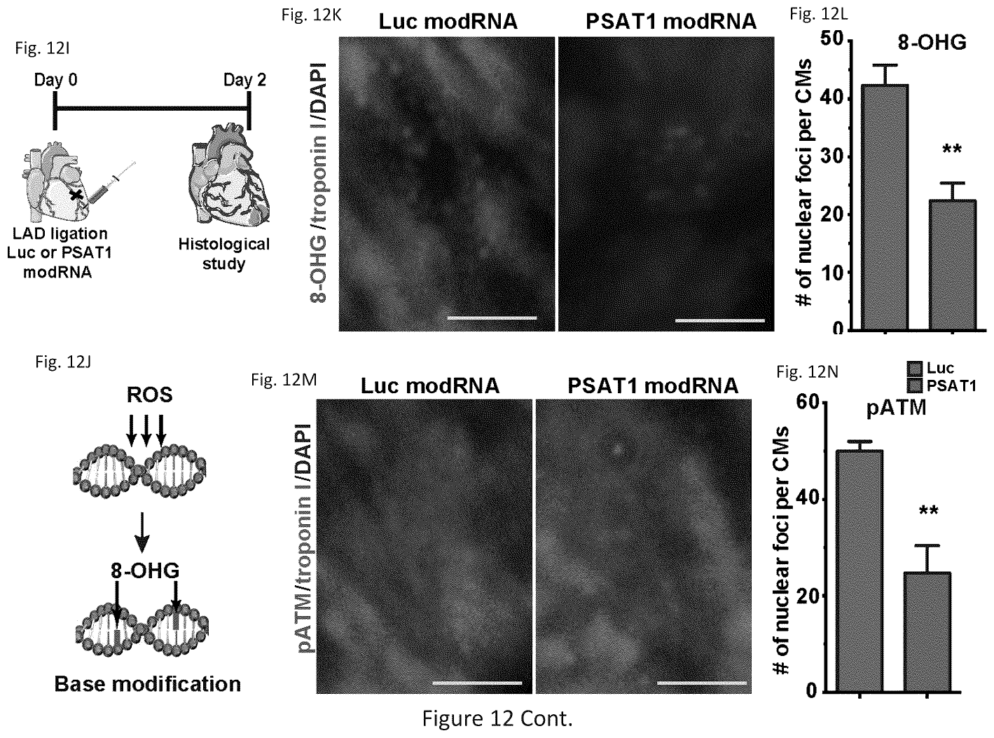

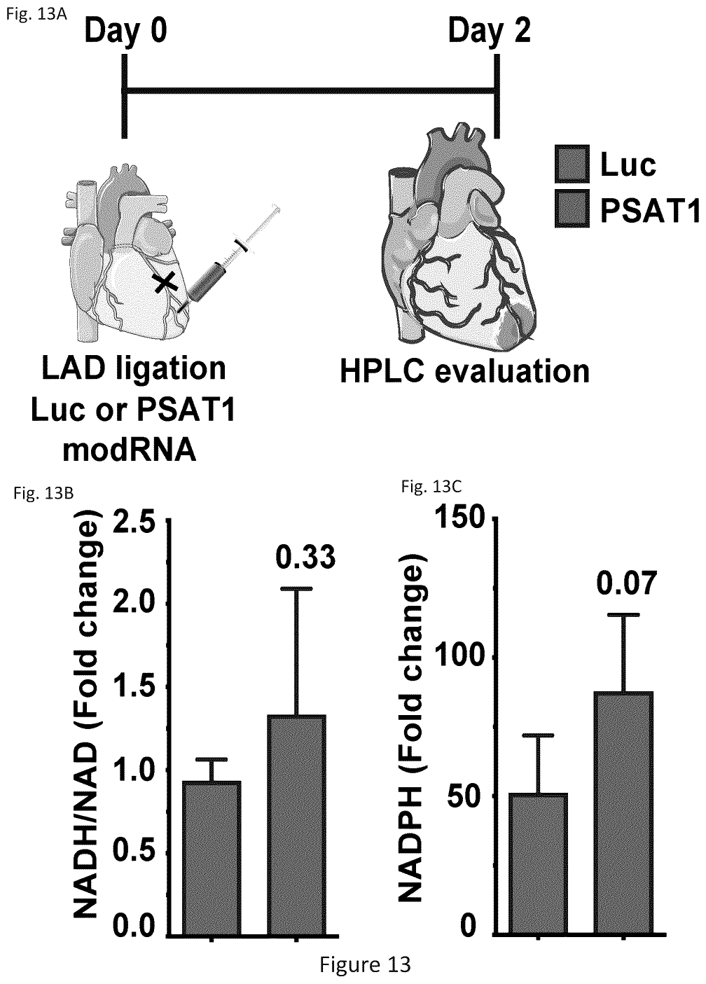

Millions of CMs die after MI due to oxidative stress, and to investigate whether PSAT1 may inhibit CM apoptosis, a mouse model of MI was used and PSAT1 modRNA was delivered (FIG. 12A). It was found that PSAT1 significantly decreased TUNEL⁺ CMs 2- and 7-days post-MI (DPMI; FIGS. 12B and 12C). It has been reported that increased oxidative stress and the DNA damage response in mice hearts induces CM apoptosis and inhibits the CM cell cycle (Puente, B. N., et al., 2014, Cell, 157:565-579). To analyze the effect of PSAT1 on oxidative stress post-MI, HPLC measurements of oxidative stress (GSH/GSSG ratio), superoxide, and other ROS were performed. The data showed PSAT1 significantly increased the GSH/GSSG ratio and reduced the levels of superoxide and other ROS post-MI (FIGS. 12D-12G). To investigate whether PSAT1 modRNA expression has an impact on the levels of reducing agents (NADPH and NADH) in CMs, their levels were analyzed by HPLC (in vivo) and ¹³C glucose flux (in vitro, NRVMs). It was found that levels of both NADPH and NADH were increased after delivery of PSAT1 modRNA post-MI (FIGS. 13A-13C).

To study the DNA damage response post-MI and modRNA injection, 8-OHG (8-oxo-7,8-dihydroguanine [8-oxoG) was used to quantify oxidative base modification of DNA and pATM (phosphorylated ataxia telangiectasia mutated) mediator of DNA damage response activation markers. Immunostaining analysis of heart sections showed significantly lower staining for the nuclear 8-OHG and pATM (nuclear foci per CMs) two days post-MI after myocardial delivery of PSAT1 modRNA compared to Luc (FIGS. 12A-12F). It has previously been shown before that a postnatal increase in mitochondrial-derived ROS induces CM cell cycle arrest through activation of the DNA damage response pathway in mouse heart within seven days of birth. It has also been demonstrated that inhibition of oxidative stress, or ROS, helps to induce the CM cell cycle or inhibit the CM apoptosis (Puente, B. N., et al., 2014, Cell, 157:565-579). This suggests that ROS-mediated activation of the DNA damage response is a critical upstream event that mediates cell-cycle arrest not only during mouse heart development but also induces deleterious cardiac remodeling in ischemic heart diseases.

### YAP1-PSAT1-β-Catenin Molecular Axis Induces CM Proliferation

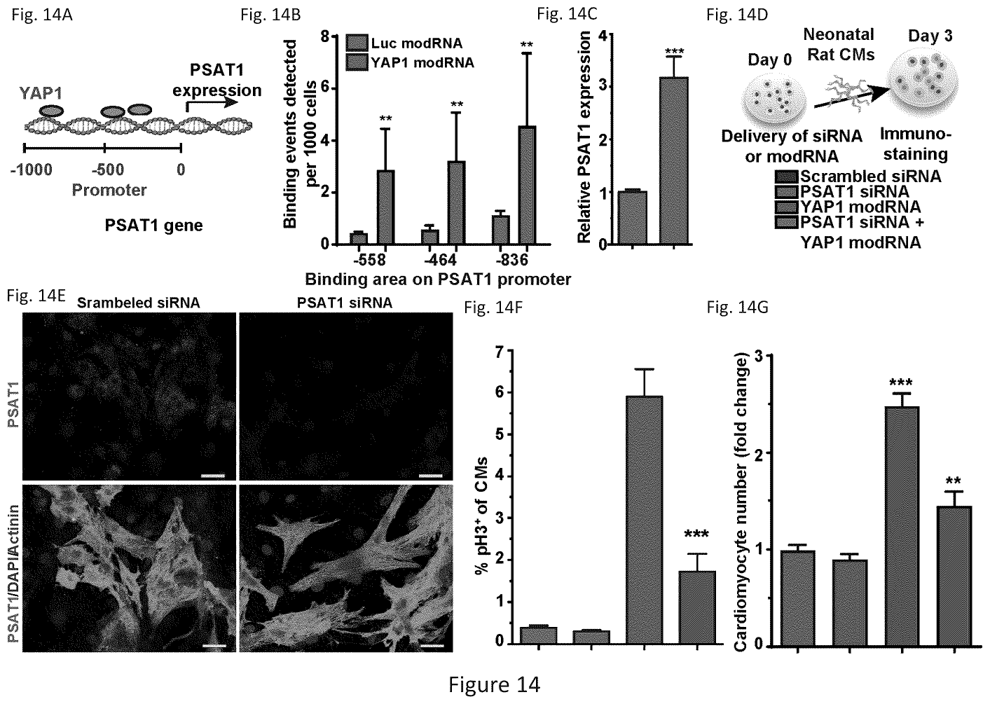

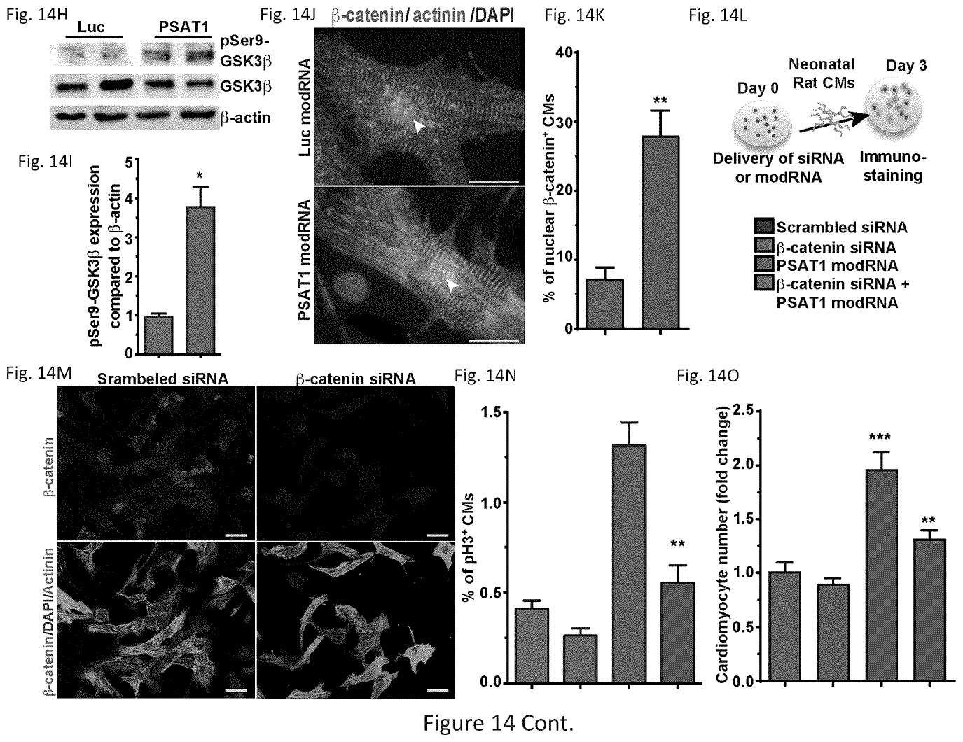

YAP1 has been shown to be a master regulator of CM proliferation and cardiac regeneration post-MI and in the heart failure mouse model. Recently, it was shown that YAP1 induces the expression of PSAT1 mRNA in tumors (Yang, C. S., et al., 2018, EMBO Reports, 19 (6): e43577). To analyze the role of YAP1 in PSAT1 expression in CMs, we treated NRVM with YAP1 modRNA and found that YAP1 modRNA significantly upregulated PSAT1 mRNA expression (FIG. 14C). To determine whether YAP1 directly binds to the promoter region of a PSAT1 to increase its transcription or if this regulation is indirect, NRVMs were treated with the YAP1 modRNA and ChIP-qPCR was performed (FIGS. 14A and 14B). It was found that YAP1 binds to the different PSAT1 promoter sites (at −836 bp, −558 bp, and −464 bp of PSAT1 promoter) and significantly induces PSAT1 mRNA expression in CMs (FIGS. 14B and 14C). To see whether PSAT1 inhibition affects the YAP1 induced CM cell cycle, NRVMs were treated with PSAT1 siRNA alone or in the presence of YAP1 modRNA (FIG. 14D). It was found that PSAT1 siRNA significantly inhibits the CM cell cycle (pH3⁺ CMs) and CM number in the presence of YAP1 modRNA, demonstrating that YAP1 induced CM cell cycle is inhibited by knockdown of PSAT1. This suggests that YAP1 requires PSAT1 to exert its effect on CM proliferation (FIGS. 14D-14G).

Nuclear β-catenin and its interaction with YAP1 in the nucleus is known to induce CM proliferation and cardiac regeneration (Heallen, T., et al., 2011, Science, 332:458-461). However, how inhibition of the hippo pathway or activation of YAP1 brings β-catenin to the nucleus in CMs is not well studied. Therefore, the role of PSAT1 modRNA expression on the Wnt pathway, specifically on β-catenin, was investigated. It was found that PSAT1 modRNA expression in NRVM induces phosphorylation of GSK3β at Ser9, an upstream regulator of β-catenin (FIGS. 14H and 14I). The increased Ser9P-GSK3β allows β-catenin to stabilize in the cytoplasm without undergoing proteolytic degradation (Cross, D. A., et al., 1995, Nature, 378:787-789). To see whether the stabilized β-catenin will translocate to the nucleus, immunostaining was performed, and it was found that the PSAT1 modRNA significantly induced β-catenin stabilization and stabilized β-catenin translocated into the CM nuclei (FIGS. 14J and 14K). To study whether β-catenin inhibition affects the PSAT1 induced CM cell cycle, CMs were treated with β-catenin siRNA alone or in the presence of PSAT1 modRNA. It was found that β-catenin inhibition significantly inhibits the CM cell cycle (pH3⁺ CMs) and CM number in the presence of PSAT1 modRNA, suggesting that the involvement of β-catenin in PSAT1 induced CM cell cycle (FIGS. 14L-14O). Taken together, this suggests that YAP1 binds to the promoter of PSAT1 (YAP1 transactivates PSAT1) and induces its expression or PSAT1 modRNA delivery activates CM cell cycle by modulating cell cycle genes and stabilizing β-catenin by GSK3β inhibition and translocation of β-catenin into the nucleus to interact with available YAP1 (Heallen, T., et al., 2011, Science, 332:458-461).

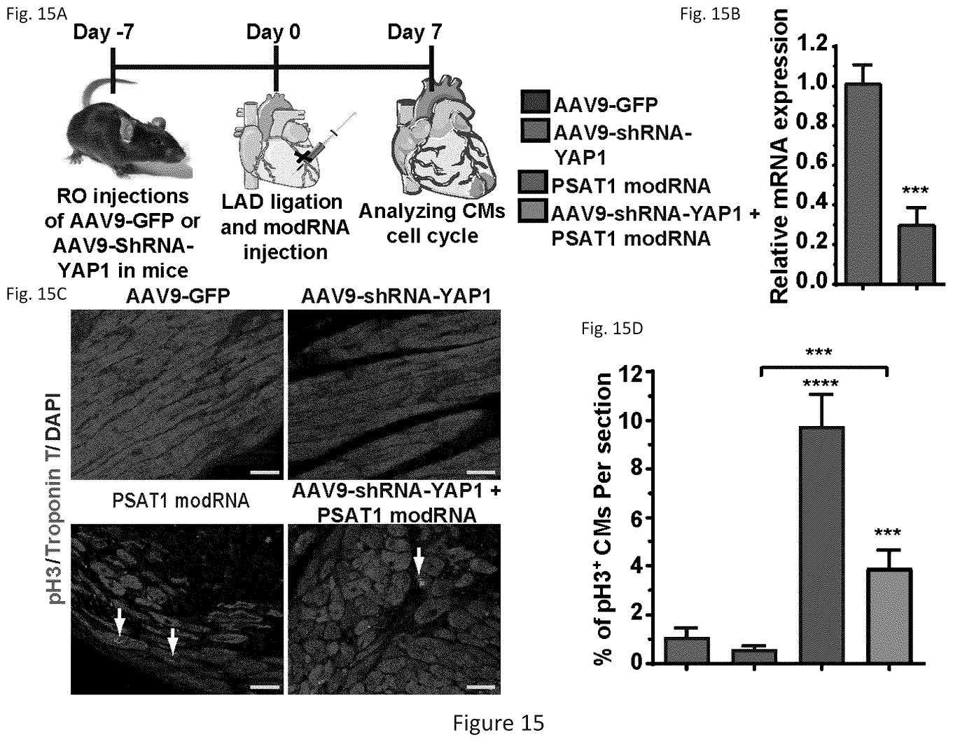

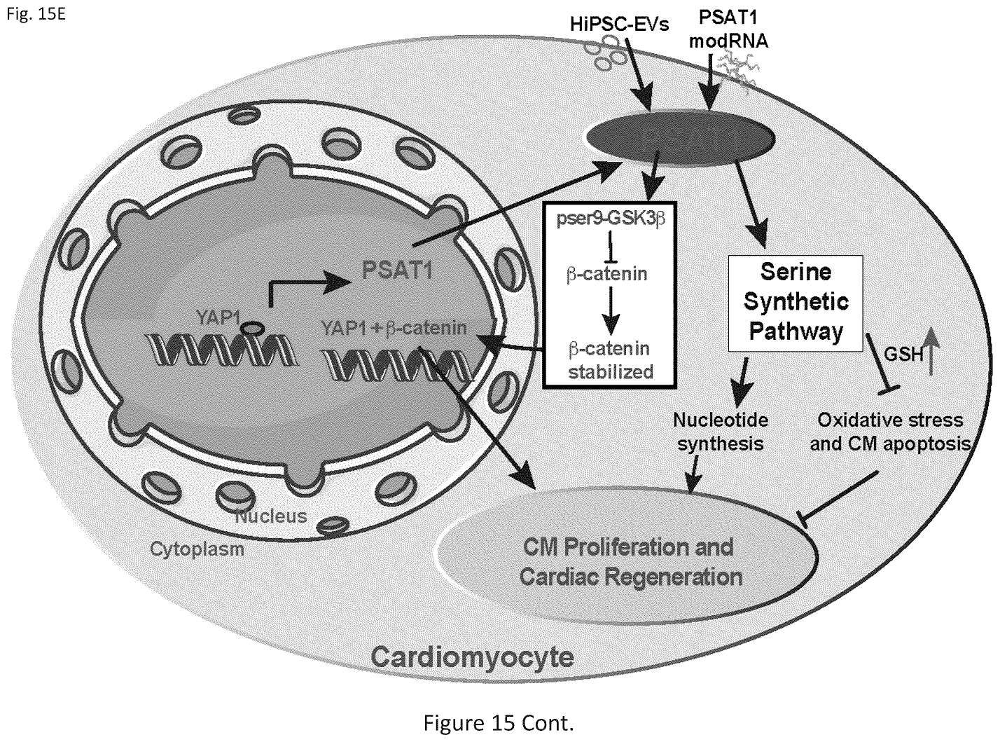

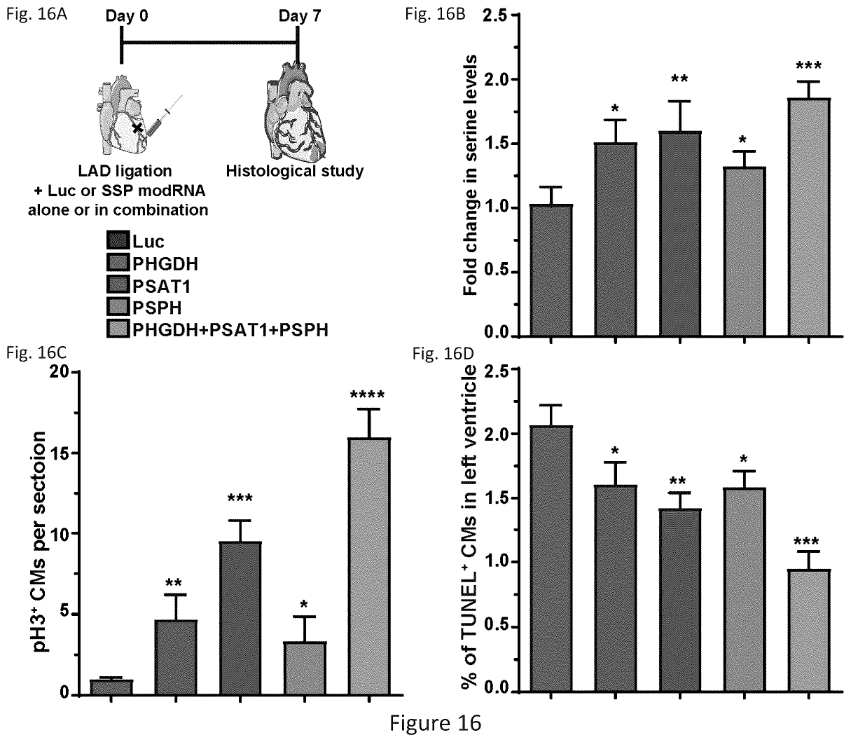

To investigate whether YAP1 inhibition affects the PSAT1 induced CM cell cycle in heart post-MI, AAV9-GFP or AAV9-YAP1-shRNA was retro-orbitally injected seven days before LAD ligation and PSAT1 modRNA delivery to infarcted myocardium (FIG. 15A-15D). Seven days after LAD and modRNA injection, it was found that AAV9-shRNA-YAP1 significantly inhibited YAP1 expression in the heart and the CM cell cycle (pH3⁺ CMs) at the basal level. PSAT1 modRNA expression under YAP1 inhibition partially reversed the CM cell cycle inhibition compared to the controls, but at significantly lower rate compared to PSAT1 modRNA alone (FIGS. 15C and 15D). These data indicate that the knockdown of YAP1 inhibits PSAT1 induced CM cell cycle suggesting the importance of the YAP1-PSAT1 molecular axis and YAP1-β-catenin interaction in nucleus to exert its effect on CM proliferation (FIG. 15E).

In summary, PSAT1 has been identified as a novel protein expressed early in mouse heart development that decreases in its expression in adult mouse hearts. While PSAT1 overexpression induces CM proliferation and cardiac regeneration, its expression in CMs under physiological condition is regulated transcriptionally by a master regulator of cardiac regeneration, YAP1. Single myocardial delivery of PSAT1 modRNA induces CM proliferation, cardiac regeneration, angiogenesis, and functional improvement and inhibits CM apoptosis, oxidative stress, DNA damage post-MI by inducing serine and nucleotide synthesis and β-catenin translocation to the nucleus. PSAT1 modRNA induces multiple processes (pleiotropic effects) which promote favorable cardiac remodeling post-MI. Homozygous mutation in PSAT1 is known to cause death before weaning in mice, and mutations also lead to development of Neu-Laxova syndrome and phosphoserine aminotransferase deficiency (Acuna-Hidalgo, R., et al., 2014, American Journal of Human Genetics, 95:285-293; Hart, C. E., et al., 2007, 80:931-937; Skarnes, W. C., et al., 2011, Nature, 474:337-342). While maintaining PSAT1 levels was found to be essential for mESC self-renewal and pluripotency, in cancer cell proliferation, it was pointed out that PSAT1 regulates cell proliferation through Wnt/catenin pathway and is regulated by ATF4 (Hwang, I. Y., et al., 2016, Cell Metabolism, 24:494-501; Gao, S., et al., 2017, Journal of Experimental & Clinical Cancer Research, 36:179).

In the mammalian heart, the metabolic switch occurs in the first week after birth when the heart utilizes more fatty acid oxidation than glycolysis as an energy source. As a result, increased oxygenation and ROS generation in the heart results in the inhibition of cardiac regeneration in neonatal mice (Puente, B. N., et al., 2014, Cell, 157:565-579). The mechanisms that regulate this metabolic switch in the mammalian heart remain unclear. Similarly, substantial metabolic shifts towards glycolytic metabolism from fatty acid oxidation occur in response to abnormal heart conditions like ischemia, hypertrophy, and pressure overload to protect against the damage and expression of fetal gene program, including genes involved in glycolytic metabolism (Puente, B. N., et al., 2014, Cell, 157:565-579; Bae, J., et al., 2021, Frontiers in Cardiovascular Medicine, 8:702920).

Catabolic reactions like lipid oxidation and oxidative phosphorylation sustain energy production for homeostasis for highly energetic organs like the heart. While dividing, cells need to acquire an adequate amount of biomass to support the production of new cells through anabolic pathways, like the synthesis of new proteins, lipids, carbohydrates, DNA, and RNA molecules. Glucose and glutamine are the key in sustaining active metabolic pathways like glycolysis and anaplerotic flux of the TCA cycle in mammalian cells (Amelio, I., et al., 2014, Trends in Biochemical Sciences, 39:191-198). In cells, serine is biosynthesized through a three-step enzymatic reaction. First, 3-phosphoglycerate that came from glycolysis will be oxidized into phosphohydroxypyruvate (pPYR) by phosphoglycerate dehydrogenase (PHGDH). Sequentially, pPYR is catalyzed by phosphoserine aminotransferase (PSAT1) to generate phosphoserine (pSER), which is then dephosphorylated by 1-3-phosphoserine phosphatase (PSPH) to form serine. External serine, and serine derived from glycolysis, can be converted to glycine by hydroxymethyltransferase (SHMT), which activates one-carbon metabolism (Amelio, I., et al., 2014, Trends in Biochemical Sciences, 39:191-198). Both serine and glycine provide the essential precursors for synthesizing proteins, nucleic acids (purines), and lipids crucial to homeostasis (Amelio, I., et al., 2014, Trends in Biochemical Sciences, 39:191-198). Glycine is a precursor for glutathione (GSH), a potent antioxidant that protects cells from oxidative stress by neutralizing ROS. The inhibition of ROS or oxidative stress has arisen as a significant element in regenerative response in mouse hearts during development and adult mice after heart injury (Puente, B. N., et al., 2014, Cell, 157:565-579). PSAT1 modRNA induced anabolic shift is conductively connected to CM proliferation. De novo serine biosynthesis from the glycerate-3P is significant to this shift and is needed for CM proliferation beneath these states.

¹³C-Glucose isotopic tracers for metabolic flux analysis showed PSAT1 modRNA significantly induced SSP and glycine levels in CMs, which results in increased levels of GSH, suggesting a more reducing environment. ROS can cause cellular oxidative stress and results in damage to proteins, lipids, and nucleic acids (Bae, J., et al., 2021, Frontiers in Cardiovascular Medicine, 8:702920). As a result of SSP activation through PSAT1 modRNA, reduced ROS levels and increased GSH to GSSG ratio are observed in mice hearts post-MI. The reduced-to-oxidized glutathione ratio is broadly used as an indicator of oxidative stress. Oxidative stress or ROS induction after MI results in the widespread activation of DNA damage or DNA damage response. In the analysis of 8-OHG and pATM, the markers of DNA damage response after PSAT1 modRNA delivery to the heart were significantly reduced in CMs (Puente, B. N., et al., 2014, Cell, 157:565-579). Recently it was shown that increased DNA damage is known to induce senescence and cell death (Yang, J. H., et al., 2023, 186:305-326). As a result of activated SSP, increased glycine and GSH levels and decreased DNA damage by PSAT1 modRNA result in a significant reduction in CM apoptosis 2- and 7-days post-MI. Activation of SSP and glycine levels also induces the synthesis of purine nucleotides, a prerequisite for the cell to enter the S-phase of the cell cycle. The increased nucleotides after PSAT1 modRNA delivery allowed CMs to enter the cell cycle and use available nucleotides for the S-phase.

Furthermore, the delivery of the serine synthesis intermediate enzymes phosphoglycerate dehydrogenase (PHGDH) and phosphoserine phosphatase (PSPH), in the form of modRNA, partially induced the CM cell cycle and inhibited CM apoptosis post-MI. At the same time, combinatorial delivery of PSAT1, PHGDH, and PSPH modRNA to mice hearts showed higher serine levels in the heart, significantly induced CM cell cycle, and inhibited CM apoptosis post-MI, suggesting the prominent role of SSP. These data link the SSP to cardiac regeneration, providing direct evidence that PSAT1-induced enhancement of SSP is required for metabolic reprogramming of CMs, generating metabolites for anabolic pathways, and increase in reducing agents to reverse oxidative stress to promote CMs to undergo cell cycle and myocardial regeneration.

The hippo pathway inhibition is known to induce CM proliferation and cardiac regeneration by activating the Wnt/β-catenin signaling pathway and the interaction of YAP1 and β-catenin in the nucleus, but it is unknown how hippo inhibition or YAP1 overexpression in the heart or CMs bring β-catenin into the nucleus (Heallen, T., et al., 2011, Science, 332:458-461; Xin, M., et al., 2013, Proceedings of the National Academy of Sciences USA, 110:13839-13844; von Gise, A., et al., 2012, Proceedings of the National Academy of Science USA, 109:2394-2399; Leach, J. P., et al., 2017, Nature, 550:260-264). First, ChiP-qPCR analysis showed that YAP1 is a trans-activator of PSAT1. Second, PSAT1 modRNA induces stabilization of β-catenin and its translocation to the nucleus by phosphorylation of Ser9 of GSK-3B, an upstream regulator of β-catenin. This results in the interaction of YAP1 and β-catenin in the nucleus, which is a prerequisite for YAP1-induced CM proliferation. To study whether PSAT1 is upstream of YAP1-β-catenin interaction and plays a vital role in regulating YAP1-β-catenin interaction in the nucleus and CM cell cycle, YAP1 or β-catenin were inhibited using AAV-shRNA YAP1 or β-catenin siRNA in CMs respectively. Both YAP1 and β-catenin inhibition in CMs markedly suppresses the PSAT1-induced CM cell cycle post-MI and iNRVMs, respectively. This data suggests that the PSAT1 regulates the YAP1-β-catenin function in the nucleus by bringing β-catenin into the nucleus, resulting in its interaction with YAP1 to induce CM proliferation program. Overall, this suggests that at the molecular level PSAT1 is a link between YAP1 and β-catenin in inducing robust CM proliferation and cardiac regeneration post-MI.

These results demonstrate a novel mechanism by which an amino acid synthesis enzyme (PSAT1) induces CM proliferation, through the activation of multiple processes, and provides a strategy for cardiac regeneration. Studies of serine synthesis pathway integration with other metabolic pathways (glycolysis, TCA, PPP, and fatty acid metabolism) and their regulation by master regulators of cardiac regeneration (PSAT1, YAP1, and β-catenin) are ongoing. This data, coupled with past reports, indicates that a modRNA approach can be used for gene expression in the mouse heart, and the single administration of specific modRNA is sufficient to illicit regenerative response in the heart post-MI (Magadum, A., et al., 2017, Cell Research, 27:1002-1019; Magadum, A., et al., 2018, Molecular Therapy-Nucleic Acids, 13:133-143; Zangi, L., et al., 2013, Nature Biotechnology, 31:898-907).

The materials and methods employed are described herein.

### Mice:

All animal procedures were performed under protocols approved by the Temple University Animal Care and Use Committee. Male and female C57BL6 mice were used. Different modRNAs (100 μg/heart) were injected directly into the myocardium in open chest surgery. Three to ten animals used for each experiment. For mice survival, C57BL6 mice (10-12-weeks-old) treated with Luc or PSAT1 modRNAs (n=10) post induction of MI and were followed for the indicated time. Deaths were monitored and documented over time. Tamoxifen-inducible CM-restricted MADM mice (α-MHC-MADM mice) mice were generated by crossing MADM mice (Jackson laboratory) to B7FVB (129)-A1cf.^{Tg(Myh6-cre/Esr1*)} 1Jmk/J (Cat no. 005657) mice to get α-MHC-MADM mice (Yuko, A. E., et al., 2021, Redox Biology, 47:102162). Tamoxifen (Sigma-Aldrich) was dissolved in sesame oil at 10 mg/ml as stock solution. To induce Cre activation, tamoxifen was administered to mice by intraperitoneal (IP) injection five times (48 h interval between administrations) at 40 mg/kg body weight for adult mice (Yuko, A. E., et al., 2021, Redox Biology, 47:102162). The tissues were harvested at mentioned time for analysis.

Synthesis of modRNA:

ModRNAs were transcribed in vitro from plasmid templates (see complete list of open reading frame sequences used to make the modRNA for this study in Table 1 below). using a customized ribonucleotide blend of cap analog, CleanCap (Cap1) (6 mM, TriLink Biotechnologies), guanosine triphosphate (1.5 mM, Life Technology), adenosine triphosphate (7.5 mM, Life Technology), cytidine triphosphate (7.5 mM, Life Technology) and N1-Methylpseudouridine-5′-Triphosphate (7.5 mM, TriLink Biotechnologies) as described previously in a recent protocol paper (Magadum A, et al., Adv Sci (Weinh), 2021, 8 (10): 2004661; Kaur K, et al., Mol Ther. 2021, 29 (10): 3042-3058). The mRNA was purified using an RNA isolation kit (Qiagen) and quantitated by Nanodrop (Thermo Scientific), precipitated with ethanol and ammonium acetate, and resuspended in 10 mM TrisHCl, 1 mM EDTA.

TABLE 1
Open reading frame sequences used for modRNA production

| Gene | Open Reading Frame |
|---|---|
| PSAT1 | atggaagccaccaagcaagtggttaactttgggcccgggcctgccaagctgccacactcggtattgttgga |
| DNA | gatccagaagcagctactagactacagaggactcggcatcagtgtgctcgaaatgagtcacaggtcatcag |
| (SEQ ID | attttgccaagattattggcaatacagagaatcttgtgagggaattgctagctgttcccaacaactacaaggtg |
| NO: 1) | atctttgtacaaggaggtgggtctggccagttcagtgctgtccccttaaatctgattggcctgaaagctggaag |
| | gagtgctgactacgtggtgaccggagcttggtcagctaaggctgcggaagaagccaagaagtttggaacg |
| | gtgaacattgtccaccctaaacttggaagttacacaaaaattccagacccaagcacctggaacctcaacccg |
| | gacgcctcctatgtatacttctgtgcaaacgagactgtgcacggggtggagtttgacttcgtacctgacgtcaa |
| | gggagcggtgctggtctgtgacatgtcctcaaacttcttatccaggccggtggatgtttccaagtttggtgtgat |
| | tttcgctggtgctcagaagaatgttggctctgccggggtgacggtggtgattgtccgggatgacctgctggg |
| | gttctcgctcagagagtgcccatcagtccttgactacaaagtgcaggctgggaacaactctttgtataacaca |
| | cctccgtgcttcagcatctacgtcatgggcatggtcctggaatggatcaagaacaacggggggccgcagc |
| | catggagaagctcagctccatcaaatcccaaatgatttatgagatcattgataattctcaaggattttatgtatgc |
| | ccagtggagcgccagaatagaagcaggatgaacatcccatttcgcattggcaacgccaaaggagacgaa |
| | gctttggaaaagcggtttcttgacaaggcggtagaactcaacatgatctccttgaaggggcacaggtcagtg |
| | ggaggcattcgtgcctctctgtataacgctgtcacaaccgaagacgttgagaagctggcggccttcatgaag |
| | aatttcttggagatgcatcagctgtga |
| PSAT1 | AUGGAAGCCACCAAGCAAGUGGUUAACUUUGGGCCCGGGCCUG |
| RNA | CCAAGCUGCCACACUCGGUAUUGUUGGAGAUCCAGAAGCAGCU |
| (SEQ ID | ACUAGACUACAGAGGACUCGGCAUCAGUGUGCUCGAAAUGAGU |
| NO: 2) | CACAGGUCAUCAGAUUUUGCCAAGAUUAUUGGCAAUACAGAGA |
| | AUCUUGUGAGGGAAUUGCUAGCUGUUCCCAACAACUACAAGGU |
| | GAUCUUUGUACAAGGAGGUGGGUCUGGCCAGUUCAGUGCUGUC |
| | CCCUUAAAUCUGAUUGGCCUGAAAGCUGGAAGGAGUGCUGACU |
| | ACGUGGUGACCGGAGCUUGGUCAGCUAAGGCUGCGGAAGAAGC |
| | CAAGAAGUUUGGAACGGUGAACAUUGUCCACCCUAAACUUGGA |
| | AGUUACACAAAAAUUCCAGACCCAAGCACCUGGAACCUCAACC |
| | CGGACGCCUCCUAUGUAUACUUCUGUGCAAACGAGACUGUGCA |
| | CGGGGUGGAGUUUGACUUCGUACCUGACGUCAAGGGAGCGGUG |
| | CUGGUCUGUGACAUGUCCUCAAACUUCUUAUCCAGGCCGGUGG |
| | AUGUUUCCAAGUUUGGUGUGAUUUUCGCUGGUGCUCAGAAGA |
| | AUGUUGGCUCUGCCGGGGUGACGGUGGUGAUUGUCCGGGAUG |
| | ACCUGCUGGGGUUCUCGCUCAGAGAGUGCCCAUCAGUCCUUGA |
| | CUACAAAGUGCAGGCUGGGAACAACUCUUUGUAUAACACACCU |
| | CCGUGCUUCAGCAUCUACGUCAUGGGCAUGGUCCUGGAAUGGA |
| | UCAAGAACAACGGCGGGGCCGCAGCCAUGGAGAAGCUCAGCUC |
| | CAUCAAAUCCCAAAUGAUUUAUGAGAUCAUUGAUAAUUCUCAA |
| | GGAUUUUAUGUAUGCCCAGUGGAGCGCCAGAAUAGAAGCAGG |
| | AUGAACAUCCCAUUUCGCAUUGGCAACGCCAAAGGAGACGAAG |
| | CUUUGGAAAAGCGGUUUCUUGACAAGGCGGUAGAACUCAACAU |
| | GAUCUCCUUGAAGGGGCACAGGUCAGUGGGAGGCAUUCGUGCC |
| | UCUCUGUAUAACGCUGUCACAACCGAAGACGUUGAGAAGCUGG |
| | CGGCCUUCAUGAAGAAUUUCUUGGAGAUGCAUCAGCUGUGA |
| PSAT1 | MEATKQVVNFGPGPAKLPHSVLLEIQKQLLDYRGLGISVLEMSHOU |
| AA | RSSDFAKIIGNTENLVRELLAVPNNYKVIFVQGGGSGQFSAVPLNLIG |
| (SEQ ID | LKAGRSADYVVTGAWSAKAAEEAKKFGTVNIVHPKLGSYTKIPDPS |
| NO: 3) | TWNLNPDASYVYFCANETVHGVEFDFVPDVKGAVLVCDMSSNFLS |
| | RPVDVSKFGVIFAGAQKNVGSAGVTVVIVRDDLLGFSLRECPSVLD |
| | YKVQAGNNSLYNTPPCFSIYVMGMVLEWIKNNGGAAAMEKLSSIK |
| | SQMIYEIIDNSQGFYVCPVERQNRSRMNIPFRIGNAKGDEALEKRFL |
| | DKAVELNMISLKGHOURSVGGIRASLYNAVTTEDVEKLAAFMKNF |
| | LEMHQL |
| Luc DNA | atggccgatgctaagaacattaagaagggccctgctcccttctaccctctggaggatggcaccgctggcga |
| (SEQ ID | gcagctgcacaaggccatgaagaggtatgccctggtgcctggcaccattgccttcaccgatgcccacattg |
| NO: 4) | aggtggacatcacctatgccgagtacttcgagatgtctgtgcgcctggccgaggccatgaagaggtacggc |
| | ctgaacaccaaccaccgcatcgtggtgtgctctgagaactctctgcagttcttcatgccagtgctgggcgccc |
| | tgttcatcggagtggccgtggcccctgctaacgacatttacaacgagcgcgagctgctgaacagcatgggc |
| | atttctcagcctaccgtggtgttcgtgtctaagaagggcctgcagaagatcctgaacgtgcagaagaagctg |
| | cctatcatccagaagatcatcatcatggactctaagaccgactaccagggcttccagagcatgtacacattcg |
| | tgacatctcatctgcctcctggcttcaacgagtacgacttcgtgccagagtctttcgacagggacaaaaccatt |
| | gccctgatcatgaacagctctgggtctaccggcctgcctaagggcgtggccctgcctcatcgcaccgcctgt |
| | gtgcgcttctctcacgcccgcgaccctattttcggcaaccagatcatccccgacaccgctattctgagcgtgg |
| | tgccattccaccacggcttcggcatgttcaccaccctgggctacctgatttgcggctttcgggtggtgctgatg |
| | taccgcttcgaggaggagctgttcctgcgcagcctgcaagactacaaaattcagtctgccctgctggtgcca |
| | accctgttcagcttcttcgctaagagcaccctgatcgacaagtacgacctgtctaacctgcacgagattgcctc |
| | tggcggcgccccactgtctaaggaggtgggcgaagccgtggccaagcgctttcatctgccaggcatccgc |
| | cagggctacggcctgaccgagacaaccagcgccattctgattaccccagagggcgacgacaagcctggc |
| | gccgtgggcaaggtggtgccattcttcgaggccaaggtggtggacctggacaccggcaagaccctggga |
| | gtgaaccagcgcggcgagctgtgtgtgcgcggccctatgattatgtccggctacgtgaataaccctgaggc |
| | cacaaacgccctgatcgacaaggacggctggctgcactctggcgacattgcctactgggacgaggacgag |
| | cacttcttcatcgtggaccgcctgaagtctctgatcaagtacaagggctaccaggtggccccagccgagctg |
| | gagtctatcctgctgcagcaccctaacattttcgacgccggagtggccggcctgcccgacgacgatgccgg |
| | cgagctgcctgccgccgtcgtcgtgctggaacacggcaagaccatgaccgagaaggagatcgtggactat |
| | gtggccagccaggtgacaaccgccaagaagctgcgcggcggagtggtgttcgtggacgaggtgcccaa |
| | gggcctgaccggcaagctggacgcccgcaagatccgcgagatcctgatcaaggctaagaaaggcggca |
| | agatcgccgtgtaa |
| PSPH | Atggtctcccactcagagctgaggaagctcttctgttcagcggatgcagtgtgctttgatgttgat |
| DNA | agcaccgtcatcagagaagaaggaatcgatgagctggccaaattctgtggtgtggaggccg |
| (SEQ ID | cagtgtctgaaatgacacggagagccatgggaggagcattgccttttaaagacgcgctcactc |
| NO: 5) | agcgcctggcactgatccagccctccagggatcaagtccagaggctcctagctgagcaccc |
| | gccacatctgactcctggcataagggagctggtaagccgcctccaggagcgtaatgtccagg |
| | tgttcctcatctctggtggctttcggagcattgtggagcacgttgctgcaaagctcaatatcccaa |
| | caaccaatgtgtttgccaataggctgaagttctactttaatggtgagtacgcaggttttgatgagat |
| | gcagccgacagccgagtcgggtgggaaaggaaaggttattcggtttttaaaggaaaaatttca |
| | ctttaagaaaataatcatgattggagatggagctacggacatggaagcctgtcctcctgctgat |
| | gctttcattggctttggaggcaatgtgatcaggcagcaggttaaggacaacgccaagtggtac |
| | atcactgacttcgtggagctgctgggagaactggaggagtga |
| PHGDH | Atggccttcgcaaatctgcgcaaagtactcatcagtgacagcctggacccctgctgccggaa |
| DNA | gatcctgcaagatggagggctgcaggtggtggagaagcagaacttgagcaaggaggagct |
| (SEQ ID | gatagctgaactccaggactgtgaaggcctcattgtccggtctgctactaaggtcactgctgatg |
| NO: 6) | tcatcaatgcagcagagaagctccaggtggtgggcagggctggcacaggtgtggacaatgt |
| | ggatctggaggctgccacgaggaagggcatcctagtcatgaacacccccaacgggaacag |
| | cctcagtgctgcggagctcacctgtgggatgatcatgtgcctggccaggcagattccccaggc |
| | aacagcttcgatgaaagatggcaaatgggaccggaagaagttcatggggacagaactgaa |
| | cgggaagacactgggaattcttggcctcggcagaattggaagagaggtggccacccgaatg |
| | caatcctttggaatgaagactgtaggctatgaccccatcatctctcctgaagtcgccgcctccttt |
| | ggtgttcagcagctgccgctggaggagatctggcctctctgtgactttataactgtccatacccca |
| | ctcctgccctctaccacaggcttgctgaatgacagcacctttgctcagtgcaagaaaggtgtgc |
| | gagtagtgaactgtgctcgaggaggcattgtggatgaaggtgccctgctgcgtgccctgcagtc |
| | tggtcagtgtgctggggctgcactggatgtgtttacagaagagccaccacgggaccgggcctt |
| | agtggaccacgagaatgtcatcagctgtccccacctgggtgccagcaccaaggaagcccag |
| | agccgctgtggggaggaaatcgcagtccagtttgtggacatggtgaaggggaaatctctaac |
| | aggcgttgtgaacgcccaggccctcaccagtgccttctctccacacaccaagccttggattggt |
| | ctggcagaagcaatgggcacgctgatgcacgcctgggctggctcccctaaagggaccatcc |
| | aggtggttacacaaggaacatctctgaagaatgctgggacctgcctgagccctgcagtcattgt |
| | cggccttctgagagaagcatctaagcaggcagacgtgaacttggtgaacgctaagctactggt |
| | gaaagaggctggcctcaatgtcaccacctcccacaaccctggggttccaggggagcagggt |
| | agcggggaatgcctcctgactgtggccctggcaggtgctccctaccaagccgtgggcttggtc |
| | cagggcaccacaccaatgctgcagatgctcaacggagctgtcttcagaccagaggtgccact |
| | acgcaggggccaacccctgctcgtattccgggctcagccctccgaccctgggatgctgccca |
| | ctatgattggcctcctggcagaggcgggtgtacagctgctgtcctaccaaacctccatggtgtct |
| | gacggagagccctggcacgtcatgggcctgtcctccctgctgcccagcctggaaacatggaa |
| | gcagcatgtattggaggctttccagttctgcttctga |

### Modrna Transfection:

In vivo transfection of modRNA was done as described previously in a recent method paper (Magadum A., et al., Circulation. 2020, 141 (15): 1249-1265; Sultana N., et al., Mol Ther, 2017, 25 (6): 1306-1315) using sucrose citrate buffer containing 10 μl of sucrose in nuclease-free water (0.3 g/ml), 10 μl of citrate (0.1M pH-7; Sigma) mixed with 10 μl of different concentrations of modRNA in saline to a total volume of 30 μl. Immediately after LAD ligation, mice received an intra-myocardial injection of modRNA (100 μg) in total volume of 30 μL at 3 different sites (mid-anterior×2 places and apical anterior) in the peri-infarct area (Magadum A., et al., Circulation. 2020, 141 (15): 1249-1265; Magadum, A., et al., 2021, Advanced Science, 9:2004661). For in vitro transfection, RNAiMAX transfection reagent (Life Technologies) was used according to manufacturer's recommendation (Magadum A., et al., Circulation. 2020, 141 (15): 1249-1265; Sultana N., et al., Mol Ther, 2017, 25 (6): 1306-1315).

### Mouse MI Model and Histology:

All surgical and experimental procedures with mice were performed in accordance with protocols approved by Institutional Animal Care and Use Committees at Temple University Animal Care and Use Committee (IACUC). C57BL6 or CM-specific MADM mice (8-12-weeks-old) were anesthetized with isoflurane. MI was induced by permanent ligation of the LAD, as previously described (Garikipati, V. N. S., et al., 2019, Nature Communications, 10:4317). Briefly, the left thoracic region was shaved and sterilized. After intubation, the heart was exposed through a left thoracotomy. A suture was placed to ligate the LAD. The thoracotomy and skin were sutured closed in layers. Excess air was removed from the thoracic cavity, and the mouse was removed from ventilation when normal breathing was established. In order to determine the effect of modRNA on cardiovascular outcome after MI, modRNAs (100 μg/heart) were injected into the infarct zone immediately after LAD ligation (Magadum A., et al., Circulation. 2020, 141 (15): 1249-1265). For examination of heart histology, hearts were collected at the end of each study, quickly washed in PBS, weighed, and fixed in 4% PFA for cryo-sectioning and immunostainings. In all experiments, the surgeon was blinded to the treatment group. For assessment of heart histology, hearts were collected at the end of each study. The hearts were excised, briefly washed in PBS, perfused with perfusion buffer, weighed and fixed in 4% PFA at 4° C. overnight. The heart blocks were transverse sectioned at 6-8 μm using cryostat. The heart sections were further processed for histological evaluation using immunostaining (see below) or histological scar staining using Masson's trichrome staining kit (Sigma) according to standard procedures. Measuring the ratio of heart weight to body weight was done using a scale. This ratio was calculated as the heart tissue weight relative to the mouse total body weight in grams (g).

### Cardiomyocyte Specific Mosaic Analysis (α-MHC-MADM Mice):

To analyze the cardiomyocyte division in vivo post-MI, cardiomyocyte specific mosaic analysis with double markers (MADM) mice, generated as described previously, were used (Yuko, A. E., et al., 2021, Redox Biology, 47:102162). The CM-specific transgenic mice were generated by crossing MADM mice (Jackson laboratory) to B6.FVB (129)-A1cf.^{Tg(Myh6-cre/Esr1*)} 1 Jmk/J (Cat no. 005657) mice to get α-MHC-MADM mice. In the MADM system, because of interchromosomal Cre-loxP recombination after S phase of the cell cycle, the daughter cells will get labeled (single colored) with either GFP (Green) or RFP (Red). The MADM single-labeling can only be attained by end of the cell cycle through cytokinesis. The inducible MADM system warrants temporal control of recombination and offers direct evidence for cell division. The α-MHC driven MADM mice allow one to analyze the CM division carefully without the interference of non-CM proliferation and non-CM-CM cell fusion. In acute MI setting, 8-12-week-old α-MHC-MADM mice were injected immediately after MI with Luc or PSAT1 modRNA. To evaluate the % single color of total labeled (Cre activated cells) CMs, mice were harvested after 4 weeks post-MI and immunostained.

### Cardiac YAP1 Knockdown:

Mice were injected with AAV9-GFP or AAV9-YAP1-shRNA (GeneCopoeia) through retro-orbital injection 7 days before LAD. A total of 5×10¹¹ viral genomes (50 μl total volume delivered) was injected per mouse. The level of YAP1 inhibition was determined at the mRNA level by qPCR 14 days post injection.

Immunostaining of Heart Sections Following modRNA Treatment:

Mice hearts were harvested, weighed, and fixed in 4% PFA/PBS overnight on shaker and then washed with PBS for 1 hour and incubated in 30% sucrose/PBS at 4° C. for 12-18 hours. Hearts were then fixed in OCT and frozen at −80° C. (Magadum A., et al., Circulation. 2020, 141 (15): 1249-1265). Transverse heart sections (6-8 μm) were made using a cryostat. Frozen sections were rehydrated in PBS for 5-10 min followed by permeabilization with PBS with 0.1% Triton™ X100 (PBST) for 7-8 min. Heart sections were then treated with 3% H₂O₂ for 5 min and washed (3×5 min) with PBST. The samples were then blocked with PBS+10% Donkey normal serum+0.1% Triton™ X100 (PBSST) for 1 hour at room temperature. Primary antibodies (see complete list of primary antibodies used for this Example in Table 2 below) diluted in PBST were added and incubated overnight at 4° C. Slides were washed with PBST (3×5 min) followed by incubation with a secondary antibody (Invitrogen, 1:200) diluted in PBST for 90-120 min at room temperature. The samples were further washed with PBST (3×5 min) and stained with DAPI or Hoechst 33342 (1 μg/ml) diluted in PBST for 7 min. After being washed with PBST (3×7 min) and tap water (1×5 min) slides were mounted with mounting medium (VECTASHIELD®) for imaging (Magadum A., et al., Circulation. 2020, 141 (15): 1249-1265). Stained slides were stored at 4° C. All staining was performed on 4-8 hearts/group, with 2-3 sections/heart. For immunostaining with wheat germ agglutinin (WGA) for CM size quantification, images were captured at 40× magnification, and ImageJ was used to determine the area of each cell. Quantitative analyses involved counting of multiple fields from 5-8 mouse hearts per group, and 3-4 sections/heart (˜50 cells per field assessed, for a total ˜250 cells per sample). For BrdU immunostaining, BrdU (1 mg/ml, Sigma) was added to the drinking water of adult mice (2-3-month-old) for 7 days before harvesting the hearts. BrdU positive CMs were counted in multiple fields from three independent samples per group and 3 sections/heart. The total number of CMs counted was ˜1-1.5×10³ CMs per section. TUNEL immunostaining of heart sections was performed according to manufacturer's recommendations (In-Situ Cell Death Detection Kit, Fluorescein, Cat #11684795910, Roche). For Immunostaining of NRVMs following modRNA treatment, modRNA-transfected neonatal CMs were fixed on coverslips with 3.7% PFA for 15 min at room temperature, permeabilized with 0.5% Triton™ X in PBS for 10 min at room temperature, and blocked with 5% normal Donkey serum+0.5% Tween™ 20 (polysorbate 20) for 30 minutes. Coverslips were incubated with primary antibodies in a humid chamber for 1 hour at room temperature followed by incubation with

TABLE 2
Antibodies

| Antigen | Dilution | Company | Catalog number |
|---|---|---|---|
| PSAT1 | 1:100 | Invitrogen | PA5-22124 |
| BrdU | 1:200 | Abcam | ab6326 |
| Aurora B | 1:200 | BD transduction | 611082 |
| α-Actinin | 1:100 | Abcam | ab9465 |
| Ki67 | 1:100 | Abcam | ab16667 |
| pH3 | 1:100 | Millipore | 06-570 |
| Troponin I | 1:50 | Santa Cruz Biotechnology | SC-15368 |
| GFP | 1:500 | Abcam | ab13970 |
| CD31 | 1:100 | R&D Biosystems | AF3628 |
| OHG | 1:100 | Abcam | ab62623 |
| WGA | 1:50 | Life technology | W11261 |
| β-catenin | 1:200 | cell signaling | 9582 |
| DsRed | 1:200 | Living Colors | 632496 |
| Troponin I | 1:200 | Abcam | Ab47003 |
| GAPDH | 1:1000 | cell signaling | 8884 |

corresponding secondary antibodies conjugated to Alexa Fluor 488 and Alexa Fluor 555 and Hoechst 33342 staining for nuclei visualization (all from Invitrogen) (Magadum A., et al., Circulation. 2020, 141 (15): 1249-1265). The fluorescent images were taken on Zeiss fluorescent microscopy at 10×, 20×, and 40× magnification.

### Western Blot Analysis:

Total protein was isolated from the respective cells or tissues at given time points. In brief, equal amounts of protein were resolved by SDS-PAG Electrophoresis system in 4%-15% Mini-PROTEAN TGX stain-free gels (Bio-Rad) and blotted onto nitrocellulose membranes (Bio-Rad). The membranes were blocked in Intercept® (TBS) blocking buffer (LI-COR) diluted 1:1 with PBS for 1 hour at room temperature and washed with PBST (1% Tween® 20, pH 7.4) for 10 min. Primary antibodies diluted in blocking buffer were added and incubated overnight at 4° C. Anti-PSAT1 (1:1,000, Invitrogen, #PA5-22124), anti-Flotillin 1 (1:1,000, Cell Signaling, #3253), anti-pSer9GSK3B (1:1,000, Cell Signaling, #9336), anti-GSK3B (1:1,000, Cell Signaling, #9315), and anti-β-actin (1:1000, Santa Cruz, #sc-47778) antibodies were used. The next day, membranes were washed with PBST (3×10 min). Anti-rabbit and anti-mouse IRDye 800CW secondary antibodies (LI-COR) were applied for 1 hr at room temperature. The membranes were washed with PBST (3×10 min) and antigen or antibody complexes were visualized and quantified using Odyssey Fc Imaging System (LI-COR Biosciences, model number 2800).

### Exosome and EV Isolation and Characterization:

Exosomes or EVs were collected from EV-free FBS media of hiPSC or IMR-90 cultured cells and isolated by ultracentrifugation method as previously described (Yue, Y., et al., 2020, Circulation Research, 126:315-329). In brief, cell debris was removed by centrifugation at 13,000 rpm (20,000×g) for 30 min followed by a 30% sucrose cushion step at 35,000 rpm (120,000×g) for 60 min (Ti50.2 rotor, Beckman Coulter, USA). The interface was further spun at 32,000 rpm (120,000×g) for 60 min to pellet EVs. The purified EVs were dissolved in PBS, aliquoted, and stored at −80° C. After isolation, EV particle number and size were characterized by Nanosight (NS300, Malvern) using Nanoparticle Tracking Analysis (NTA) software.

### Proteomics (Mass Spectrometry):

Exosomes or EVs isolated from hiPSC and IMR-90 were solubilized in lysis buffer (8 M urea and 1% protease inhibitor cocktail). Protein concentration was determined with a BCA kit according to the manufacturer's instructions. Equal amounts of protein from the two groups were digested according to manufacturer's instructions. The peptides were fractionated by high pH reverse-phase High-Performance Liquid Chromatography (HPLC) and dried by vacuum centrifugation. The peptides were injected into the mass spectrometer according to standard procedure and data was collected. The proteins were quantified using Spectronaut™ proteomics software (DIA) and statistical analysis was performed using Perseus. T-tests were used to identify upregulated or downregulated peptides or proteins between the samples. Biological function and protein analysis were performed using gene ontology and KEGG.

### Exosome or EVs Injection:

Mice underwent surgery to ligate the left anterior descending coronary artery as reported previously, followed by intramyocardial administration of exosomes or EVs (1×10⁹) from hiPSC cells and IMR-90 cells suspended in PBS into the left ventricular wall (border zone) at two different locations and at the heart apex at one location immediately after left anterior descending ligation (Yue, Y., et al., 2020, Circulation Research, 126:315-329). The saline group underwent the same surgery but received saline without exosomes. Tissue was harvested at 7- or 28-days post-MI for histological analysis.

### Chromatin Immunoprecipitation:

Neonatal rat cardiomyocytes were transfected with Luc or YAP1 modRNA, fixed with 1% formaldehyde for 15 min after 24 hours, quenched with 0.125 M glycine, and sent to Active Motif Services (Carlsbad, CA) to be processed for ChIP-qPCR. In brief, chromatin was isolated by the addition of lysis buffer, followed by disruption with a Dounce homogenizer. Lysates were sonicated and the DNA sheared to an average length of 300-500 bp. Genomic DNA (Input) was prepared by treating aliquots of chromatin with RNase, proteinase K, and heat for de-crosslinking, followed by SPRI bead clean up (Beckman Coulter). Pellets were resuspended and the resulting DNA was quantified on a ClarioStar spectrophotometer (BMG Labtech). Extrapolation to the original chromatin volume allowed quantitation of the total chromatin yield.

An aliquot of chromatin (30 μg) was precleared with protein A agarose beads (Invitrogen). Genomic DNA regions of interest were isolated using 5 μg of antibody against YAP1 (US Biological, catalog number Y1200-01D). Complexes were washed, eluted from the beads with SDS buffer, and subjected to RNase and proteinase K treatment. Crosslinks were reversed by incubation overnight at 65° C. and ChIP DNA was purified by phenol-chloroform extraction and ethanol precipitation.

Quantitative PCR (qPCR) reactions were carried out in triplicate on specific genomic regions using SYBR™ Green Supermix (Bio-Rad, Cat #170-8882) on a CFX Connect™ Real Time PCR system (see primer sequence in Table 3 below). The resulting signals were normalized for primer efficiency by carrying out QPCR for each primer pair using Input DNA.

TABLE 3
Primer Sequences for ChIP-qPCR

| Gene | Forward | Reverse |
|---|---|---|
| Negative control | aggcacataggaggtaaaagttc | ggaggtcacaggaggacttc |
| | (SEQ ID NO: 7) | (SEQ ID NO: 8) |
| Positive control | ttggttctgtcggatgtctg | ttggttctgtcggatgtctg |
| (Cand1-126) | (SEQ ID NO: 9) | (SEQ ID NO: 10) |
| rPSAT1-558 | catcctcccgagtgagtaatg | gggtgaagaaagggctaaag |
| | (SEQ ID NO: 11) | (SEQ ID NO: 12) |
| rPSAT1-464 | catttgttccagagccagtc | ttctttgggtgccttgtctc |
| | (SEQ ID NO: 13) | (SEQ ID NO: 14) |
| rPSAT1-836 | ccaggacagccactctcaag | gcacaagagccaagattcag |
| | (SEQ ID NO: 15) | (SEQ ID NO: 16) |

### RNA Interference:

For siRNA knockdown, CMs were transfected 48 to 72 hours after seeding by Lipofectamine® RNAiMAX kit (cationic transfection reagent, Invitrogen) with validated siRNAs or AllStars Negative Control siRNA (Qiagen) (100 nM), and washed after 5 hours. Gene expression inhibition was verified by antibody staining after 72 hours. siRNA sequences: β-catenin-5′-UAGUCGUGGGAUCGCACCCUG-3′ (SEQ ID NO: 17), PSAT1-5′-AUGUCCAUGACGUAGAUGC-3′ (SEQ ID NO:18), YAP1-5′-AAAGGGAUCUGAACUAUUG-3′ (SEQ ID NO:19).

### RNA Isolation and Gene Expression Profiling Using Real-Time PCR:

Total RNA was isolated using TRIzol™ RNA isolation and RNeasy® (Qiagen) then reverse transcribed with High-Capacity cDNA Reverse Transcription Kit (Applied Biosystems), according to the manufacturer's instructions. Real-time qPCR analyses were performed on a StepOnePlus™ Real-Time PCR System using SYBR™ Green (Applied Biosystems). The primer sequences were synthesized by IDT (see Table 4 below). Data were normalized to 18s expression, where appropriate (endogenous controls). Fold-changes in gene expression were determined by the DOCT method and were presented relative to an internal control.

TABLE 4
Primer Sequences for qPCR gene expression profiling

| Gene | Forward | Reverse |
|---|---|---|
| PSAT1 | caagcacctggaacctcaac | caccagcgaaaatcacacca |
| | (SEQ ID NO: 20) | (SEQ ID NO: 21) |
| cMyc | aggcagctctggagtgagag | cctggctcgcagattgtaag |
| | (SEQ ID NO: 22) | (SEQ ID NO: 23) |
| Cdc20 | ttcgtgttcgagagcgatttg | accttggaactagatttgccag |
| | (SEQ ID NO: 24) | (SEQ ID NO: 25) |
| Cdk1 | tttcggccttgccagagcgtt | gtggagtagcgagccgagcc |
| | (SEQ ID NO: 26) | (SEQ ID NO: 27) |
| Ccnd2 | gtcacccctcacgacttcat | ttccagttgcaatcatcgac |
| | (SEQ ID NO: 28) | (SEQ ID NO: 29) |
| Ccnb1 | aaggtgcctgtgtgtgaacc | gtcagccccatcatctgcg |
| | (SEQ ID NO: 30) | (SEQ ID NO: 31) |
| 18s | agtccctgccctttgtacaca | cgatccgagggcctcacta |
| | (SEQ ID NO: 32) | (SEQ ID NO: 33) |
| P27 | aggagagccaggatgtcagc | cagagtttgcctgagacccaa |
| | (SEQ ID NO: 34) | (SEQ ID NO: 35) |
| P21 | gacaagaggcccagtacttc | gcttggagtgatagaaatctgtc |
| | (SEQ ID NO: 36) | (SEQ ID NO: 37) |

### Neonatal Rat and Fetal or Adult Mouse CMs Isolation:

CMs from 2-3 day-old neonatal rat's (Sprague Dawley) hearts were isolated as previously described (Magadum A., et al., Circulation. 2020, 141 (15): 1249-1265). Multiple rounds of digestion with collagenase II (0.14 mg/mL, Invitrogen) were used. After each digestion, the supernatant was collected in horse serum (Invitrogen). Total cell suspension was centrifuged at 300× g for 5 min. Supernatants were discarded, and cells were resuspended in DMEM (GIBCO) with 0.1 mM ascorbic acid (Sigma), 0.5% Insulin-Transferrin-Selenium (100×), penicillin (100 U/mL) and streptomycin (100 μg/mL). Cells were plated in plastic culture dishes for 90 min until most of the non-myocytes attached to the dish and myocytes remained in suspension. Myocytes were then seeded at 1×10⁵ cells/well in a 24 well plate. Isolated CMs were incubated for 48 hours in DMEM containing 5% horse serum plus cytarabine (Ara c). After incubation, cells were transfected with different doses of different modRNAs, or siRNAs, as described in the text. PH755, an inhibitor of phosphoglycerate dehydrogenase, was used at 10 μM to study the SSP.

### ¹³C Isotopic Tracers for Metabolic Flux Analysis:

P3 neonatal rat CMs (2×10⁶) were cultured in DMEM and transfected with Luc or PSAT1 modRNA. After 6 hours medium was changed with ¹³C glucose-containing DMEM. Samples were collected at 10 min, 2 hours and 18 hours by flash freezing the cell culture plates using liquid nitrogen and stored at −80° C. Metabolites were extracted from cells using 80% methanol (Magadum A., et al., Circulation. 2020, 141 (15): 1249-1265). Targeted LC/MS analyses were performed on a Q Exactive Orbitrap mass spectrometer (Thermo Scientific) coupled to a Vanquish UPLC system (Thermo Scientific). The Q Exactive was operated in polarity-switching mode. A Sequant ZIC-HILIC column (2.1 mm i.d.×150 mm, Merck) was used for separation of metabolites with a flow rate was 150 μL/min. Buffers consisted of 100% acetonitrile for A, and 0.1% NH₄OH/20 mM ammonium acetate in water for B. The solvent gradient ran from 85 to 30% A in 20 min followed by a wash with 30% A and re-equilibration at 85% A. Metabolites were identified my exact mass (>5 ppm) and standard retention times. Relative metabolite quantitation was performed based on peak area for each metabolite. All data analyses were done using scripts written in house (Metabolomics Facility, Biotechnology Resource Center, Cornell University).

### HPLC Measurements of ROS:

ROS production in heart tissue was measured using HPLC for dihydroethidium (DHE) oxidation products (Magadum A., et al., Circulation. 2020, 141 (15): 1249-1265). Immediately after harvesting hearts, a 20 mg segment of heart tissue was cut into small pieces and incubated with DHE (100 μM) in PBS containing DTPA (100 μM) at 37° C. for 30 min. The sample was washed with PBS/DTPA, extracted with acetonitrile (500 μl) and briefly sonicated (3× 30 sec, 8W). After centrifugation (13,000×g, 10 min at 4° C.), the supernatant was collected and dried under vacuum, and pellets stored at −20° C. in the dark until analysis. Samples were resuspended in 120 μl PBS-DTPA and injected into the HPLC system. The superoxide-specific DHE oxidation product 2-hydroxyethidine (EOH) was quantified by comparison of the peak signal between samples and standard solutions under identical chromatographic conditions, and expressed as EOH/mg tissue.

### HPLC Measurements of Glutathione:

The level of glutathione (GSH) in heart tissue was measured by an HPLC method with electrochemical detection, as previously described (Magadum A., et al., Circulation. 2020, 141 (15): 1249-1265). Heart samples were homogenized in a 100 mM acetate buffer (pH 5.4, containing 10 μM DTPA). A small aliquot was used to quantify total protein. The remaining sample was deproteinized with 10% TCA, centrifuged at 10,000×g for 5 min at 4° C., the supernatant filtered (0.45 μm) and stored at −80° C. Samples were injected onto a C18 column (Phenomenex C18, 3 μm, 150×4.6 mm) and eluted at 0.5 ml/min using an isocratic mobile phase solution (25 mM NaH₂PO₄, 1 mM 1-octane sulfonic acid, 6% acetonitrile, pH 2.6, H₂O with phosphoric acid). GSH levels were quantified by comparison to standards subjected to the same HPLC conditions. Glutathione disulfide (GSSG) levels were indirectly determined by its enzymatic reduction with glutathione reductase (0.6 U/ml plus 0.2 mg/ml NADPH) for 30 min. The reaction was stopped by adding 5% TCA on ice. The GSH levels were normalized by the amount of protein. GSSG quantification was achieved by the difference between total reduced GSSG to total GSH in each sample.

### Serine Level Analysis:

Heart tissue (˜10 mg) or cultured NRVMs (˜1×10⁶ cells) were rapidly homogenized on ice with 100 ml of ice-cold serine assay buffer. The homogenates were centrifuged at 15,000×g for 10 minutes at 4° C. and the supernatant transferred to a new microfuge tube. Analysis was performed according to the utilized kit and fluorescence of all samples was recorded at an excitation wavelength of 535 nm and an emission wavelength of 587 nm in endpoint mode.

### Statistical Analysis:

Statistical significance was determined by unpaired two-tailed t-test, one-way ANOVA, or Bonferroni post hoc test, or Log-rank (Mantel-Cox) test for survival curves as detailed in respective figure legends. A p value less than 0.05 was considered significant. All graphs represent average values, and values were reported as mean±standard error of the mean. Unpaired two-tailed t-tests were based on assumed normal distributions. For the quantification of the number of luminal structures (CD31), WGA, OHG, ATM or positive TUNEL, BrdU, ki67, pH3 or Aurora B CMs, the results were acquired from at least 3 heart sections per heart and number of mice as mentioned in respective figure legends.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for regenerating cardiac tissue, comprising an activator of phosphohydroxythreonine aminotransferase (PSAT1).

2. The composition of claim 1, wherein said activator of PSAT1 comprises a nucleic acid molecule encoding PSAT1.

3. The composition of claim 2, wherein said nucleic acid molecule encoding PSAT1 comprise a modified RNA molecule.

4. The composition of claim 3, wherein said modified RNA molecule comprises methylpseudouridine in place of naturally occurring uridine.

5. The composition of claim 3, wherein said modified RNA molecule comprises a codon optimized sequence for expression in mammalian cells.

6. The composition of claim 3, wherein said modified RNA molecule encoding PSAT1 comprises one or more selected from the group consisting of:
a. an RNA sequence at least 90% identical to SEQ ID NO: 2;
b. an RNA sequence at least 90% of the length of SEQ ID NO: 2;
c. an RNA sequence at least 90% identical to a nucleotide sequence at least 90% of the length of SEQ ID NO: 2; and
d. the RNA sequence of SEQ ID NO: 2.

7. The composition of claim 2, wherein said nucleic acid molecule encoding PSAT1 comprises one or more selected from the group consisting of:
a. a nucleotide sequence at least 90% identical to SEQ ID NO: 1;
b. a nucleotide sequence at least 90% of the length of SEQ ID NO: 1;
c. a nucleotide sequence at least 90% identical to a nucleotide sequence at least 90% of the length of SEQ ID NO: 1; and
d. the nucleotide sequence of SEQ ID NO: 1.

8. The composition of claim 7, wherein said nucleic acid molecule comprises a plasmid vector.

9. The composition of claim 8, wherein said plasmid vector comprises a plasmid vector optimized for expression in mammalian cells.

10. The composition of claim 1, wherein said activator of PSAT1 comprises a PSAT1 polypeptide.

11. The composition of claim 10, wherein said PSAT1 polypeptide comprises one or more selected from the group consisting of:
a. an amino acid sequence at least 90% identical to SEQ ID NO: 3;
b. an amino acid sequence at least 90% of the length of SEQ ID NO: 3;
c. an amino acid sequence at least 90% identical to an amino acid sequence at least 90% of the length of SEQ ID NO: 3; and
d. the amino acid sequence of SEQ ID NO: 3.

12. The composition of any of the preceding claims, further comprising a pharmaceutically acceptable carrier.

13. A method of administering a composition for regenerating cardiac tissue to subject in need thereof, comprising administering to the subject the composition of claim 1.

14. A method of regenerating cardiac tissue in a subject in need thereof, comprising administering to the subject an activator of PSAT1.

15. A method of treating a disease or disorder associated with cardiac tissue damage in a subject in need thereof, comprising administering to the subject an activator of PSAT1.

16. The method of claim 15, wherein said disease or disorder comprises one or more selected from the group consisting of: fibrotic diseases, myocardial infarction, ischemic heart disease, heart failure, and dilated cardiomyopathy (DCM).

17. The method of claim 16, wherein said activator of PSAT1 is administered via intra-myocardial injection.

18. The method of claim 17, wherein said intra-myocardial injection is localized to the peri-infarct area of the infarcted heart.

19. The method of claim 15, wherein the subject is a mammal.

20. The method of claim 19, wherein the subject is a human.

21. The method of claim 15, wherein the gene expression of PSAT1 increases after administration of the activator of PSAT1.

22. The method of claim 21, wherein the gene expression of PSAT1 increases by at least 10-fold one day after administration of the activator of PSAT1.

23. A method for inducing/reactivating proliferation of neonatal or adult cardiomyocytes in vitro or in vivo following myocardial infarction (MI).

24. A method for inducing or activating the serine synthesis pathway (SSP) of neonatal or adult cardiomyocytes in vitro or in vivo following myocardial infarction, comprising administering an activator of PSAT1.

25. A method for inhibiting oxidative stress and reactive oxygen species in neonatal or adult cardiomyocytes in vitro or in vivo following myocardial infarction, comprising administering an activator of PSAT1.

26. A method for inhibiting cardiomyocyte apoptosis in neonatal or adult cardiomyocytes in vitro or in vivo following myocardial infarction, comprising administering an activator of PSAT1.

27. A method for stabilizing β-catenin and its translocation to the nucleus in neonatal cardiomyocytes in vitro, comprising administering an activator of PSAT1.

28. A method of promoting activation of PSAT1 by YAP1 through transactivation in neonatal cardiomyocytes in vitro, comprising administering one or more composition that promotes YAP1 binding to the promoter site of PSAT1, thereby inducing expression of PSAT1.

29. A method for inducing nucleotide synthesis in neonatal or adult cardiomyocytes in vitro or in vivo following myocardial infarction, comprising administering an activator of PSAT1.

30. A method of treating ischemic heart injury in a subject in need thereof, the method compromising administering a modified mRNA (modRNA) encoding phosphoserine aminotransferase 1 or phosphohydroxythreonine aminotransferase 1 (PSAT1) to a heart tissue of the subject.

31. The method of claim 30, wherein administering the modRNA encoding PSAT1 to heart tissue of a subject in need thereof improves heart function by at least 50% compared to an untreated subject with identical disease condition and predicted outcome.

32. The method of claim 30, wherein administering the modRNA encoding PSAT1 to heart tissue of a subject in need thereof increases life expectancy by at least 20% compared to an untreated subject with identical disease condition and predicted outcome.

33. The method of claim 30, wherein administering the modRNA encoding PSAT1 to heart tissue of a subject in need thereof reduces cardiac fibrosis by at least 50% compared to an untreated subject with identical disease condition and predicted outcome.

34. A gene delivery system for treatment of ischemic heart injury, comprising modRNA encoding PSAT1 for local administration to heart tissue.

35. The gene delivery system for treatment of ischemic heart injury of claim 34, wherein the modRNA encoding PSAT1 increases PSAT1 expression in said heart tissue.

36. The gene delivery system for treatment of ischemic heart injury of claim 34, further compromising a delivery agent.

37. The gene delivery system for treatment of ischemic heart injury of claim 36, wherein the delivery agent specifically targets hearts tissue.

38. The gene delivery system for treatment of ischemic heart injury of claim 34, wherein the gene delivery system is formulated for intracardiac injections.

39. The method of claim 15, wherein the individual is provided one or more additional therapies for the disease or disorder.

40. The method of claim 23, wherein the method comprises myocardial administering one or more modRNA selected from the group consisting of phosphoglycerate dehydrogenase (PHGDH), PSAT1, and phosphoserine phosphatase (PSPH), wherein the modRNA induces the cardiomyocyte cell cycle and inhibits cardiomyocyte apoptosis.

41. The method of claim 24, wherein activation of the SSP inhibits cardiomyocyte apoptosis and oxidative stress post-MI. The method of claim 30, wherein angiogenesis is improved after delivery of PSAT1 modRNA.

43. The method of claim 30, wherein DNA damage and/or DNA damage response was inhibited after delivery of PSAT1 modRNA.

44. The method of claim 30, wherein scar size is reduced after delivery of PSAT1 modRNA.

45. The method of claim 30, wherein the PSAT1 modRNA is encapsulated in a delivery vehicle.
